(19) United States
(12) Patent Application Publication
(10) Pub. No.: US 2024/0301178 A1
(43) Pub. Date: Sep. 12, 2024

(54) POLYSACCHARIDE COMPLEX

(71) Applicant: ACARYON GMBH, Berlin (DE)

(72) Inventors: Philippe ULSEMER, Schildow (DE); Kawe TOUTOUNIAN, Berlin (DE)

(21) Appl. No.: 18/277,535

(22) PCT Filed: Feb. 17, 2022
(86) PCT No.: PCT/EP2022/053948

§ 371 Date: Aug. 16, 2023

(30) Foreign Application Priority Data
Feb. 17, 2021 (EP) 21157724.2
Feb. 17, 2021 (LU) 102545

(51) Int. Cl. C08L 3/02; A61K 39/00

(52) U.S. Cl. CPC C08L 3/02; A61K 2039/55583

(57) ABSTRACT

The present invention relates to a polysaccharide complex comprising at least one polysaccharide (P1) and ii) a heterologous lipid carrier wherein the heterologous lipid carrier comprises a) a lipid portion comprising at least one ceramide-like glycolipid moiety and/or a fatty acid moiety and b) at least one non-lipid moiety, preferably said non-lipid portion comprises at least one carbohydrate moiety, at least one lipopeptide moiety, at least one linker, at least one chemical compound, and/or at least one peptide moiety, wherein i) and ii) are associated. Further, the invention is directed to a method to prepare said polysaccharide complex.

### TECHNICAL FIELD OF THE INVENTION

The present invention relates to a polysaccharide complex comprising at least i) one polysaccharide (P1) and ii) a heterologous lipid carrier wherein the heterologous lipid carrier comprises a) a lipid portion comprising at least one ceramide-like glycolipid moiety and/or a fatty acid moiety and b) at least one non-lipid moiety, preferably said non-lipid portion comprises at least one carbohydrate moiety, at least one lipopeptide moiety, at least one linker, at least one chemical compound, and/or at least one peptide moiety, wherein i) and ii) are associated by direct bonding to each other. Further, the invention is directed to a method to prepare said polysaccharide complex.

### BACKGROUND ART

Food graded decoys or pharmacological decoys, means nano- or microparticles, or microorganisms displaying a specific structure (e.g., oligosaccharide or peptide) on their surface have a broad potential of applications in food and medicine like oral vaccines, oral drug delivery systems and anti-infectious agents for example in the gastrointestinal tract, on the skin and other sites such urogenital tract, respiratory system or oral cavity.

Standard procedures to produce such decoys are mainly based on chemically produced nanoparticles and either microorganisms fused with a protein composed of a peptide of interest bound to a protein-domain (anchor-domain) or microorganisms genetically modified to express the structure of interest (Genetically Modified Organisms, GMOs). The use of nanoparticles may be limited by natural biodegradation and absorption as well as by their potential toxic effect in cells or organs where they may accumulate. The use of microorganisms fused with a protein composed of a peptide of interest bound to a protein-domain (anchor-domain) may be limited by i) the natural proteolytic activities of the said microorganism that may affect the stability and integrity of the fusion protein and ii) the potential immunogenic properties of the anchor-domain. Finally, the use of genetically modified microorganisms is coupled with production and safety issues, including the difficulty to produce them at pharmaceutical grade, their ability to mutate, their potential to colonize the host permanently and their potential to cause diseases.

Thus, as is evident from the above, to obtain decoys displaying one or several structure of interest (e.g., peptide or carbohydrate) to the environment, the prior art provides technologies associated with significant technological limitations and/or safety concerns.

However, such decoys presenting oligosaccharides, peptides or chemical molecules of interest to the environment would be of high interest and present broad application potential including, among others, the development of oral vaccines, oral drug delivery systems, anti-infectious agents and functional foods.

Polysaccharides are a diverse class of polymeric materials of natural (animal, plant, algal) origin formed via glycosidic linkages of monosaccharides. Natural polysaccharides can be divided from higher plants (e.g. cellulose, starch, pectin, inulin, gums), marine organisms (e.g. chitosan, alginate, agar and carrageenans), microorganisms (e.g. glucans, pullulan, dextran, curdlan, gellan, hyaluronic acid and levan) or animals (glycogen).

Starch is one of the most important polysaccharides and a major component of many food plants such as wheat, barley, rice, corn, potato, sweet potato and cassava. Starch is used in food, cosmetics, paper, textile, and certain industries, as adhesive, thickening, stabilizing, stiffening, and gelling (pasting) agents. Starch consists of amylose and branched amylopectin molecules in molar ratios of 15%-25% and 85%-75%, respectively.

Complexation between starch and lipids has been the subject of intensive research over the past 50 years and their effects on the functional properties and nutritional value of starch have been characterized.

Starch-lipid complexes, are also referred to as a resistant starch. Although many factors can influence the rate of enzymic breakdown of starch, the access of enzyme to the glucoside bonds in the substrate is a major determinant. The increased resistance of starch to enzymatic hydrolysis by lipid addition is directly related to the formation and structure of starch-lipid complexes. The greater the structural order of complexes, the more resistant the starch is to amylolysis. Higher complexation temperatures and pressures lead to more structurally ordered complexes, resulting in lower digestibility

Lipids reported to be inserted into amylose helices include fatty acids, monoglycerides and lysophospholipids. Diglycerides and triglycerides do not form complexes with starch (Wang et al., Compr Rev Food Sci Food Saf. 2020; 19:1056-1079).

Similarly, lipids form complexes with other polysaccharides. In particular for example polysaccharides such as cellulose, glycogen, chitosan (Wydro et al., Biomacromolecules, 2007 August; 8(8):2611-7), pectin (Guzman-Puyol et al., PLOS One 2015 Apr. 27; 10(4):e0124639), methylmannose (Liu et al., Structure and Stability of Carbohydrate-Lipid Interactions. Methylmannose Polysaccharide-Fatty Acid Complexes, https://doi.org/10.1002/cbic.201600123) are known to form complexes with lipids.

WO2017/193161 discloses an agent delivery system comprising a membrane encapsulating an agent. The membrane may comprise gangliosides, phospholipids and sphingolipids. The agent may be a starch-containing drug. The membrane composition forms a physical barrier around such a starch containing drug, which may be construed as associated”. However, WO/2017/193161 do not disclose a direct bonding/between the polysaccharide (starch) and a heterologous lipid carrier (i.e. gangioside). Thus, in such a “membrane encapsulated polysaccharide (i.e. starch)-construct”, the membrane forms a physical barrier which restricts the movement of such a starch-containing drug relative to the constituents of said membrane. Furthermore, in such “membrane encapsulated polysaccharide construct”, the said polysaccharide (i.e. starch) is not accessible from/to the environment, restricting direct interactions between the polysaccharide and the environment (i.e. drug release, binding of constituents etc.). Furthermore, the integrity of such “membrane encapsulated polysaccharide constructs” may be strongly impacted by external conditions known to impact the integrity of lipid membranes/liposome like conditions encountered in the intestinal tract, extreme pH, temperature etc (Liposome destruction by hydrodynamic cavitation in comparison to chemical, physical and mechanical treatments. Žiga Pandur, Iztok Dogsa, Matevž Dular, David Stopar. Ultrasonics—Sonochemistry 61 (2020) 104826).

As starch and other polysaccharides are abundant, non-toxic, biodegradable, edible, and relatively inexpensive materials, the preparation of polysaccharide-based microparticles has attracted much attention in the food and drug industries. Of special interest is its ability to resist the digestion but to be fermented in the colon. Thus, it is especially suited for the site-specific delivery of a drug to the colon.

The ability of polysaccharide to associate lipids and to form microparticles may allow the generation of a new kind of microparticles displaying one or several structure of interest (e.g., peptide or carbohydrate) to the environment.

### SUMMARY OF THE INVENTION

The present invention relates to a Polysaccharide complex comprising
i) at least one polysaccharide (P1) and
ii) a heterologous lipid carrier wherein the heterologous lipid carrier comprises
a) a lipid portion comprising at least one ceramide-like glycolipid moiety and/or a fatty acid moiety and
b) at least one non-lipid moiety, preferably said non-lipid portion comprises at least one carbohydrate moiety, at least one lipopeptide moiety, at least one linker, at least one chemical compound, and/or at least one peptide moiety, wherein i) and ii) are associated by direct bonding to each other.

Further, the invention is directed to a process for preparing a polysaccharide complex and to a polysaccharide preparable by said process.

Further, the invention relates to a composition comprising
i) one or more of the polysaccharide complex(es); preferably said composition comprising a mixture of same or different polysaccharide complex(es) and/or
ii) one or more of the polysaccharide complex(es) and at least one pharmaceutically acceptable carrier.

Further, the invention is directed to the use of the composition of claim 10 or 11 as nutraceutical.

Moreover, the invention is directed to a vaccine or adjuvant comprising the polysaccharide complex or composition.

It has been shown that the inventive polysaccharide complex is stable under physiological conditions. Thus, the present invention makes it possible to prepare polysaccharide complexes with, for example, specific carbohydrate groups which allow site specific delivery of drugs or specific carbohydrate groups or peptides which allow specific binding of pathogens and/or their toxins.

### BRIEF DESCRIPTION OF THE FIGURES

FIG. 1A: Specific binding of Cholera toxin to GM1-loaded corn starch.

FIG. 1B: Resistance of GM1-loaded corn starch to the action of bile salt.

FIG. 1C: Resistance of GM1-loaded corn starch to the action of different treatments

FIG. 2: Loading of starch with GM1 and GM1-red.

FIG. 3: Loading of Gb3 to corn starch.

FIG. 4: Fluorescence microscopy of GM1-loaded cornstarch incubated with CT-Texas-red and Stx1-Texas-red. FIGS. 4A, B and C present the microscopic observation of GM1-loaded starch incubated with CT-Tx in phase contrast, with the texas red-specific filter and as overlapping of both pictures respectively. FIGS. 4D, E and F present the microscopic observation of GM1-loaded starch incubated with Stx-Tx in phase contrast, with the texas red-specific filter and as overlapping of both pictures respectively.

### DETAILED DESCRIPTION OF THE INVENTION

The solution of the present invention is described in the following, exemplified in the appended examples, illustrated in the Figures and reflected in the claims.□

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 means less than the number indicated. Similarly, more than or greater than means more than or greater than the indicated number, f.e. more than 80% means more than or greater than the indicated number of 80%.□

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

The term “alkyl” refers to a monoradical of a saturated straight or branched hydrocarbon. Preferably, the alkyl group comprises from 1 to 80 carbon atoms, i.e., 1, 2, 3, 4, 5, 6, 7, 8, 9, 10, 11, 12, 13, 14, 15, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and 80 carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 4, i.e., 1, 2, 3, or 4, carbon-carbon double bonds. Preferably, the alkenyl group comprises from 2 to 10 carbon atoms, i.e., 2, 3, 4, 5, 6, 7, 8, 9, 10, 11, 12, 13, 14, 15, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and 80 carbon atoms. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, and the like. If an alkenyl group is attached to a nitrogen atom, the double bond cannot be alpha to the nitrogen atom.

The term “cycloalkyl” represents cyclic non-aromatic versions of “alkyl” and “alkenyl” with preferably 5 to 15 carbon atoms, such as 5 to 15 carbon atoms, i.e., 5, 6, 7, 8, 9, 10, 11, 12, 13, 14, and 15 carbon atoms, more preferably 5 to 8 carbon atoms, even more preferably 5 to 7 carbon atoms. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The term “cycloalkyl” is also meant to include bicyclic and tricyclic versions thereof. If bicyclic rings are formed it is preferred that the respective rings are connected to each other at two adjacent carbon atoms, however, alternatively the two rings are connected via the same carbon atom, i.e., they form a spiro ring system or they form “bridged” ring systems. Preferred examples of cycloalkyl include C₃-C₈-cycloalkyl, in particular cyclopropyl, cyclobutyl, cyclopentyl, cyclohexyl, cycloheptyl, cyclooctyl, spiro[3,3]heptyl, spiro[3,4]octyl, spiro[4,3]octyl, bicyclo[4.1.0]heptyl, bicyclo[3.2.0]heptyl, bicyclo[2.2.1]heptyl, bicyclo[2.2.2]octyl, bicyclo[5.1.0]octyl, and bicyclo[4.2.0]octyl.

The term “heterocyclyl” means a cycloalkyl group as defined above in which from 1, 2, 3, or 4 carbon atoms in the cycloalkyl group are replaced by heteroatoms of O, S, or N. Preferably, in each ring of the heterocyclyl group the maximum number of O atoms is 1, the maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholino, isochromanyl, chromanyl, pyrrolidinyl, imidazolidinyl, pyrazolidinyl, piperidinyl, piperazinyl, indolinyl, isoindolinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydrothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), di- and tetrahydrobenzofuranyl (1- and 2-), di- and tetrahydroindolyl, di- and tetrahydroisoindolyl, di- and tetrahydrobenzothienyl (1- and 2), di- and tetrahydro-1H-indazolyl, di- and tetrahydrobenzimidazolyl, di- and tetrahydrobenzoxazolyl, di- and tetrahydroindoxazinyl, di- and tetrahydrobenzisoxazolyl, di- and tetrahydrobenzothiazolyl, di- and tetrahydrobenzisothiazolyl, di- and tetrahydrobenzotriazolyl, di- and tetrahydroquinolinyl, di- and tetrahydroisoquinolinyl, di- and tetrahydrobenzodiazinyl, di- and tetrahydroquinoxalinyl, di- and tetrahydroquinazolinyl, di- and tetrahydrobenzotriazinyl (1,2,3- and 1,2,4-), di- and tetrahydropyridazinyl, di- and tetrahydrophenoxazinyl, di- and tetrahydrothiazolopyridinyl (such as 4,5,6-7-tetrahydro[1,3]thiazolo[5,4-c]pyridinyl or 4,5,6-7-tetrahydro[1,3]thiazolo[4,5-c]pyridinyl, e.g., 4,5,6-7-tetrahydro[1,3]thiazolo[5,4-c]pyridin-2-yl or 4,5,6-7-tetrahydro[1,3]thiazolo[4,5-c]pyridin-2-yl), di- and tetrahydropyrrolothiazolyl (such as 5,6-dihydro-4H-pyrrolo[3,4-d][1,3]thiazolyl), di- and tetrahydrophenothiazinyl, di- and tetrahydroisobenzofuranyl, di- and tetrahydrochromenyl, di- and tetrahydroxanthenyl, di- and tetrahydrophenoxathiinyl, di- and tetrahydropyrrolizinyl, di- and tetrahydroindolizinyl, di- and tetrahydroindazolyl, di- and tetrahydropurinyl, di- and tetrahydroquinolizinyl, di- and tetrahydrophthalazinyl, di- and tetrahydronaphthyridinyl (1,5-, 1,6-, 1,7-, 1,8-, and 2,6-), di- and tetrahydrocinnolinyl, di- and tetrahydropteridinyl, di- and tetrahydrocarbazolyl, di- and tetrahydrophenanthridinyl, di- and tetrahydroacridinyl, di- and tetrahydroperimidinyl, di- and tetrahydrophenanthrolinyl (1,7-, 1,8-, 1,10-, 3,8-, and 4,7-), di- and tetrahydrophenazinyl, di- and tetrahydrooxazolopyridinyl, di- and tetrahydroisoxazolopyridinyl, di- and tetrahydropyrrolooxazolyl, and di- and tetrahydropyrrolopyrrolyl. Exemplary 5- or 6-membered heterocyclyl groups include morpholino, pyrrolidinyl, imidazolidinyl, pyrazolidinyl, piperidinyl, piperazinyl, di- and tetrahydrofuranyl, di- and tetrahydrothienyl, di- and tetrahydrooxazolyl, di- and tetrahydroisoxazolyl, di- and tetrahydrooxadiazolyl (1,2,5- and 1,2,3-), dihydropyrrolyl, dihydroimidazolyl, dihydropyrazolyl, di- and tetrahydrotriazolyl (1,2,3- and 1,2,4-), di- and tetrahydrothiazolyl, di- and tetrahydroisothiazolyl, di- and tetrahydrothiadiazolyl (1,2,3- and 1,2,5-), di- and tetrahydropyridyl, di- and tetrahydropyrimidinyl, di- and tetrahydropyrazinyl, di- and tetrahydrotriazinyl (1,2,3-, 1,2,4-, and 1,3,5-), and di- and tetrahydropyridazinyl.

The term “aryl” refers to a monoradical of an aromatic cyclic hydrocarbon. Preferably, the aryl group contains 5 to 14, more preferably 5 to 10, such as 5, 6, or 10 carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl.

The invention is directed to a polysaccharide complex.

The polysaccharide complex comprises
i) at least one polysaccharide (P1) and
ii) a heterologous lipid carrier.
i) and ii) are associated.

Preferably, the polysaccharide complex comprises one polysaccharide (P1) comprising at least 50 monosaccharides, more preferably at least 100, more preferably at least 500. The polysaccharide (P1) may be a branched polysaccharide. A branched polysaccharide comprises at least two linkage types between monomers, thus monosaccharides. One of these linkages is usually more frequent in the polysaccharide (P1) than the other. The most common binding type is usually the linkage type which connects the monomers of the backbone polymer chains. Less frequent is usually the linkage type which is present due to branching. Glycogen for example comprises glucose as monomers which are linked via α-1,4 linkages in long chains. Additionally, glycogen is branched via α-1,6 linkages, which thus are less frequent than α-1,4 linkages. Therefore, the degree of branching in polysaccharides may be described by the ratio of the more frequent linkage type between the monomers and the less frequent linking type.

Preferably, the polysaccharide (P1) has a low degree of branching. More preferably, in the polysaccharide (P1) the ratio between the most frequent linking type between the monosaccharides and the less frequent binding type between monosaccharides is at least 15:1, preferably at least 50:1, more preferably at least 100:1; or there is only one binding type between the monosaccharides.

The Polysaccharide (P1) may comprise at least one polysaccharide selected from the group consisting of starch, amylose, amylopectin, cellulose, glycogen, chitosan, methylmannose; preferably the polysaccharide (P1) comprises amylose and branched amylopectin molecules, more preferably the polysaccharide (P1) comprises amylose and branched amylopectin molecules in molar ratios of 15%-25% amylose and 85%-75% branched amylopectin molecules.

The heterologous lipid carrier comprises
a) a lipid portion comprising at least one ceramide-like glycolipid moiety and/or a fatty acid moiety and
b) at least one non-lipid moiety, preferably said non-lipid portion comprises at least one carbohydrate moiety, at least one lipopeptide moiety, at least one linker, at least one chemical compound, and/or at least one peptide moiety.

In one embodiment, the at least one non-lipid moiety comprises at least one GM1 moiety.

In one embodiment, the at least one non-lipid moiety comprises at least one GM1 and at least one Gb3 moiety.

As used herein, the term “lipopeptide” may refer to a molecule comprising a peptide part (e.g. a peptide molecule as described herein) associated with (e.g. coupled to) a lipid part (e.g. a lipid molecule as described herein). Preferably said peptide part of the lipopeptide is linked directly to said lipid part of the lipopeptide via a covalent bond or a linker or linking molecule (e.g. a peptide-based linker or a chemical linker such as e.g. bi-functional linkers (NHS-ester and maleimid), copper-free click-chemistry alkyne-azido triazole linkages, unnatural amino acids, carbohydrate-mediate linkages, photocross-linkers. Other non-covalent linkages allowing an association (e.g. a stable association) between said peptide part and said lipid part are also within the scope of the present invention. The peptide of the peptide part can be of any length and may include one or more modifications, preferably post-translational modifications, including, but not limited to, glycosylation, sulfation, carboxylation, phosphorylation etc. The peptide part can also be a protein composed of one or more covalently or non-covalently associated polypeptide chains. The peptide can be covalently or non-covalently coupled to an additional chemical molecule. Each polypeptide chain may independently be modified, preferably post-translationally or chemically modified. In a preferred embodiment the peptide part is a binding molecule. Further preferably, the peptide part comprises an antibody, a fragment thereof or mutated or modified version. In another preferred embodiment the peptide can be a molecule which mimics a carbohydrate. Those molecules may comprise one or more peptide-based linkers. In another preferred embodiment the peptide part comprises a lectin or a fragment thereof. In another preferred embodiment the peptide part comprises an immunologically active molecule such as a cytokine or chemokine or a fragment thereof. In a preferred embodiment the peptide part binds to a toxin of the invention, a toxin receptor, a receptor, a cell, a protein, an immunologically active molecule, an inflammatory molecule or another molecule. The peptide part of the lipopeptide can also be any other naturally occurring, therefrom derived, or chemically synthetized chemical moiety able to bind to a toxin of the invention, a toxin receptor, a receptor, a cell, a protein, a carbohydrate or another molecule, or an immunologically active molecule. In a certain embodiment such molecules comprise DNA or RNA or a DNA or RNA peptide or protein complex. In further embodiments peptide/protein based binding molecules, which are modified to keep/stabilize their binding structure in environments like the gastrointestinal tract, Lung, urogenital tract better than the non-modified form, are used. Modifications, e.g. mutation of sites prone for proteolysis or of sites which when mutated help to stabilize the spacial three-dimensional structure, may also be used. Technologies to achieve this are known in the art. An advantage will be the generation of phage-display libraries displaying such molecules for selection of binders to toxins and/or pathogens, which is within scope of the present invention. Various lipid molecules of different type, length of fatty acid chains, with or without natural and chemical modifications may be used in the present invention. The sequence of the peptide/protein part may be designed accordingly and tested with suitable amino acids and side chains for covalent coupling. Chemical, peptide and carbohydrate-based spacers may be used for a better presentation on the bacteria. The coupling of the peptides with lipids can be for example carried on by the use of bi-functional linkers (e.g. NHS-ester and maleimid), copper-free click-chemistry alkyne-azido triazole linkages, unnatural amino acids, carbohydrate-mediate linkages, photocross-linkers a.o., which readily known in the art.

A linker according to the present invention may refer to one or more linkers and/or one or more branched linker(s). Such branched linker((s) allow(s) coupling directly or via another linker(s) of one or more moieties such as carbohydrate(s), peptide(s) or protein(s) and of a core structure as defined elsewhere herein such as GM1, a derivative thereof or a carbohydrate structure of GM1. Said linker according to the present invention may comprise one or more carbohydrate(s), peptide(s), optionally substituted (C₁-C₁₅)alkyl moiety(s), optionally substituted (C₅-C₁₀)aryl moiety(s), (C₅-C₁₅)heterocyclyl moiety(s), and/or optionally substituted (C₅-C₁₀)heteroaryl moiety(s).
i) and ii) are associated, preferably by direct bonding to each other, preferably by non-covalent bonds. Non-covalent bonds are bonds other than covalent bonds, such as hydrogen-bridge bonds or van der Waals forces/bonds. Direct bonding in this context means any form of direct interaction between i) and ii) that is mediated by covalent or non-covalent interactions, these interactions being spontaneous or induced by a chemical, enzymatical or physical process, preferably spontaneous, and resulting in i) and ii) being bound in such a manner that i) and ii) not merely restrict or influence each others movement just by their physical presence, for example by forming a physical barrier, such as a membrane, and that at least part of i) and ii) is exposed and accessible to/from the environment. The absence or presence of a membrane and/or a liposome may be determined by exposing the respective compound or complex to membrane/liposome destroying conditions as listed below and in the examples. See also Liposome destruction by hydrodynamic cavitation in comparison to chemical, physical and mechanical treatments. Žiga Pandur, Iztok Dogsa, Matevž Dular, David Stopar. Ultrasonics-Sonochemistry 61 (2020) 104826.

Preferably, the polysaccharide complex, in particular the bonding between i) and ii) is stabile against at least one of the following conditions:
a) treatment with Triton X-100 at final concentrations of 0.85 mM at room temperature for 15 min and/or
b) treatment with ethanol 10% at room temperature for 15 min and/or
c) treatment with FeCl₃ at a final concentration of 100 mM at room temperature for 15 min and/or
d) incubation at pH1 and/or pH9 at room temperature for 15 min and/or
e) vortex with glass beads at 3000 rpm for 4 min and/or
f) incubating at 75 to 85° C. for 15 min and/or
g) treatment with 0.3% bile salt for 1 hour at 37° C.

Wherein “room temperature” means a temperature in the range 19 to 24° C.

Preferably, the heterologous lipid carrier is defined according to the general formula (I) or (II);

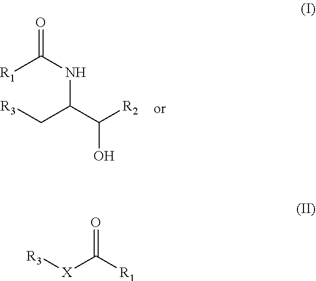

wherein
R₁ is linear or branched (C₁-C₈₀)alkyl, (C₂-C₈₀)alkenyl, preferably (C₁₀-C₅₀)alkyl, (C₁₀-C₅₀)alkenyl, more preferably (C₁₁-C₃₀)alkyl, (C₁₁-C₃₀)alkenyl, most preferably (C₁₂-C₂₅)alkyl, (C₁₂-C₂₅)alkenyl;
   or R₁ is —C(OH)—R₄, wherein R₄ is linear or branched (C₁-C₈₀)alkyl or (C₂-C₈₀)alkenyl;
   or R₁ is (C₆-C₈₀)alkyl or (C₆-C₈₀)alkenyl wherein:
(i) the (C₆-C₈₀)alkyl or the (C₆-C₈₀)alkenyl is substituted with at least one (C₅-C₁₅)cycloalkyl, (C₅-C₁₅)cycloalkenyl, (C₅-C₁₅)heterocyclyl, or (C₅-C₁₄)aryl; or
(ii) the (C₆-C₈₀)alkane or the C₆-C₈₀)alkenyl includes, within the (C₆-C₈₀)alkyl or (C₆-C₈₀)alkenyl chain, at least one (C₅-C₁₅) cycloalkyl, (C₅-C₁₅)heterocyclyl, or (C₅-C₁₄)aryl;
R₂ is —CH₂(CH₂)ₙCH₃, —CH(OH)(CH₂)ₒCH₃, —CH(OH)CH(OH)(CH₂)ₚCH₃, —CH═CHCH₂OH, —CH(OH)(CH₂)_{q}CH(CH₃)₂, —CH═CH(CH₂)ᵣCH₃, —CH(OH)(CH₂)ₛCH(CH₃)CH₂CH₃,
   n, o, p, q, r and s are independently selected from an integer ranging from 1-25, preferably 4-17.
R₃ is an α-linked or a β-linked mono, di- or polysaccharide (P2) moiety, at least one peptide moiety, or a polysaccharide peptide moiety comprising an α-linked or a β-linked mono-, di- or polysaccharide and at least one peptide;
   optionally R₃ comprises at least one linker in addition to the α-linked or the β-linked mono, di- or polysaccharide (P2) moiety, the at least one peptide moiety, or the polysaccharide peptide moiety comprising an α-linked or a β-linked mono-, di- or polysaccharide and at least one peptide;
   optionally R₃ is selected from the group consisting of: a polysaccharide which may comprise acetate groups, most preferably the first sugar of the said carbohydrate is galactose, a glucose, a mannose, a xylose, a neuraminic acid, a N-acetyl glucosamine, N-acetyl galactosamine or a galacturonic acid;

In further embodiments R₃ may be selected from
i) Galβ1 3GalNAcβ1 4(Neu5Acα2,3)Galβ1 4Glcβ1-B—, or

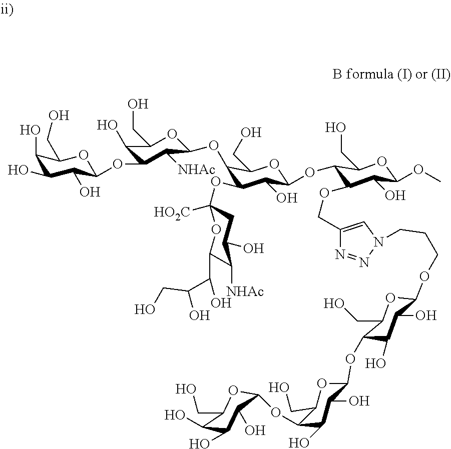

or
iii) αNeu5Ac(2-3) βDGal(1-3) βDGalNAc(1-4)[α Neu5Ac(2-3)]βDGal(1-4) βDGlc(1)-B—;
N-Acetyl-D-galactose-β-1,4-[N-Acetylneuraminidate-α-2,3-]-Galactose-β-1,4-glucose(1)-B—;
βDGalNAc(1-4)[αNeu5Ac(2-8)αNeu5Ac(2-3)]βDGal(1-4)βDGlc(1)-B—; βDGal(1-3) βDGalNAc(1-4)[αNeu5Ac(2-8)αNeu5Ac(2-3)]βDGal(1-4)βDGlc(1)-B—; βDGal(1-3)βDGalNAc(1-4)[αNeu5Ac(2-8)αNeu5Ac(2-3)]βDGal(1-4)βDGlc(1)-B—; αNeu5Ac(2-3) βDGal(1-3)βDGalNAc(1-4)[αNeu5Ac(2-8) αNeu5Ac(2-3)] βDGal(1-4) βDGlc(1)-B—;
βDGal(1-3)βDGalNAc(1-4)[αNeu5Ac(2-8)αNeu5Ac(2-8)αNeu5Ac(2-3)]βDGal(1-4) βDGlc(1)-B-Neu5Acα2,3Galβ1,3GalNAcβ1,4(Neu5Acα2,8Neu5Acα2,8Neu5Acα2,3)Galβ1,4Glcβ1-B—; Galβ1,3GalNAcβ1,4Galβ1,4Glcβ1-B—; Neu5Acα2,3Galβ1,3GalNAcβ1,4Galβ1,4Glcβ1-B—; Neu5Acα2,8Neu5Acα2,3Galβ1,4Glcβ1-B—; Neu5Acα2,3Galβ1,4Glcβ1-B—; Neu5Acα2,8Neu5Acα2,8Neu5Acα2,3Galβ1,4Glcβ1-B—; GalNAc(β1-3)Gal(α1-4)Gal(β1-4)Glc(β1)-B—; or
iv) Gal α1-3Galβ1-4GlacNAc-B—, Gal(α1-4)Gal(β1-4)GlcNAc-B—, Gal(α 1-4)Gal(β1-4)Glc-B—, NAc-(β1-3)Gal(β1-4)Glc-B—, Gal(α 1-4)Gal(β1-4)GlcNAc(β1-2) Man-B—; or
v) any of the following glycan moieties listed in column 2, titled with “Structure” of

TABLE 1
Glycan moieties

| Column 1: Example | Column 2 : Structure |
|---|---|
| 2-6 Sialyl i-Lewis x | Neu5Ac(α 2-6)Gal(β1-4)GlcNAc(β1-3)Gal(β1-4)[Fuc(α |
| | 1-3)]GlcNAc(β1-)B- |
| 3′-Sulfo Lewis a | HSO3(-3)Gal(β1-3)[Fuc(a1-4)]GlcNAc(β1-)B- |
| 3′-Sulfo Lewis x | HSO3(-3)Gal(β1-4)[Fuc(a1-3)]GlcNAc(β1-)B- |
| 6,6′-Disulfo Sialyl Lewis x | Neu5Ac(α 2-3)[HSO3(-6)]Gal(β1-4)[Fuc(α 1-3)][HSO3(- |
| | 6)]GlcNAc(β1-)B- |
| 6-Sulfo Lewis x | Gal(β1-4)[Fuc(α 1-3)][HSO3(-6)]GlcNAc(β1-)B- |
| 6′-Sulfo Sialyl Lewis x | Neu5Ac(α 2-3)[HSO3(-6)]Gal(β1-4)[Fuc(α 1- |
| | 3)]GlcNAc(β1-)B- |
| 6^{(GlcNAc)}-su-SLe^{x} | Neu5Acα2-3Galβ1-4(Fucα1-3)(6-O-Su)GlcNAcβ-B- |
| 6′-Sia-6-Su-LacNAc | Neu5Acα2-6Galβ1-4(6-O-Su)GlcNAcβ-B- |
| 6-Su-3′SiaLe^{c} | Neu5Acα2-3Galβ1-3(6-O-Su)GlcNAcβ-B- |
| 6-Su-3′SLN | Neu5Acα2-3Galβ1-4(6-O-Su)GlcNAcβ-B- |
| 3′SLN(Gc) | Neu5Gcα2-3Galβ1-4GlcNAcβ-B- |
| 6′SLN(Gc) | Neu5Gcα2-6Galβ1-4GlcNAcβ-B- |
| GlcNAcβ3’LacNAc | GlcNAcβ1-3Galβ1-4GlcNAcβ-B- |
| Isomaltotriose | Glcα1-6Glca1-6Glcβ-B- |
| Chitotriose | GlcNAcβ1-4GlcNAβ1-4GlcNAcβ-B- |
| alpha Gal epitope, | Gal(α 1-3)Gal(β1-4)GlcNAc-B- |
| Arthro | GlcNAcβ1,3Manβ1,4Glcβ-B- |
| Aₜᵣᵢ | GalNAcα1-3(Fucα1-2)Galβ-B- |
| Bₜᵣᵢ | Galα1-3(Fucα1-2)Galβ-B- |
| Gal₂3,4-GlcNAc | Galβ1-4(Galβ1-3)GlcNAcβ-B- |
| asialo-GM1, GA1 | DGal(β1-3)DGalNAc(β1-4)DGal(β1-4)DGlc(β1-1)-B- |
| asialo-GM2, GA2 | DGalNAc(β1-4)DGal(β1-4)DGlc(β1-1)-B- |
| Blood Group A Trisaccharide | GalNAc(α 1-3)[Fuc(α 1-2)]Gal(β1-)B- |
| Blood Group A Type 1 | GalNAc(α 1-3)[Fuc(α 1-2)]Gal(β1-3)GlcNAc(β1-)B- |
| Blood Group A Type 1 (difucosyl) | GalNAc(α 1-3)[Fuc(α 1-2)]Gal(β1-3)[Fuc(α 1- |
| | 4)]GlcNAc(β1-)B- |
| Blood Group A Type 2 | GalNAc(α 1-3)[Fuc(α 1-2)]Gal(β1-4)GlcNAc(β1-)B- |
| Blood Group A Type 2 (difucosyl) | GalNAc(α 1-3)[Fuc(α 1-2)]Gal(β1-4)[Fuc(α 1)- |
| | 3)]GlcNAc(β1-)B- |
| Blood Group A Type 3 | GalNAc(α 1-3)[Fuc(α 1-2)]Gal(β1-3)GalNAc(α1-)B- |
| Blood Group A Type 4 | GalNAc(α 1-3)[Fuc(α 1-2)]Gal(β1-3)GalNAc(β1-)B- |
| Blood Group B | Gal(α 1-3)[Fuc (α 1-2)]Gal(β1-3)GlcNAc(β1-3)Gal-B-, |
| Blood Group B Type 2 | Gal(α 1-3)[Fuc(α 1-2)]Gal(β1-4)GlcNAc(β1-)B- |
| Blood Group H Type 1, | Fuc(α 1-2)Gal(β1-3)GlcNAc(β1-)B- |
| Blood Group H Type 2, | Fuc(α 1-2)Gal(β1-4)GlcNAc(β1-)B- |
| Blood Group H Type 3, | Fuc(α 1-2)Gal(β1-3)GalNAc(α1-)B- |
| Blood Group H, | Fuc(α 1-2)Gal(β1-)B- |
| Sia6′H (type 2) | Neu5Acα2-6(Fucα1-2)Galβ1-4GlcNAcβ-B- |
| 6-LacNAc-TF | Galβ1-4GlcNAcβ1-6(Galβ1-3)GalNAcα-B- |
| C-Series Ganglio-sides Oligo- | -Gal(β1-3)GalNAc(β1-4)[Neu5Ac(α 2-8)Neu5Ac(α 2- |
| saccharide/Ganglio-tetraosyl | 8)Neu5Ac(α 2-3)]Gal(β1-4)Glc(β1-1)-B- |
| Core Structure | |
| C-Series Ganglio-sides Oligo- | Neu5Ac(α 2-8)Neu5Ac(α 2-8)Neu5Ac(α 2-3)Gal-B- |
| saccharide/Hemato- or Ganglio- | |
| Type | |
| Cyclic Sialyl 6-Sulfo Lewis x | cyclicNeu5Ac(α 2-3)Gal(b1-4)[Fuc(α 1-3)][HSO3(- |
| | 6)]GlcNAc-B- |
| Dimeric Lewis x | Gal(β1-4)[Fuc(α 1-3)]GlcNAc(β1-3)Gal(b1-4)[Fuc(α 1- |
| | 3)]GlcNAc(β1-)B- |
| Disialyl Lewis a | Neu5Ac(α 2-3)Gal(β1-3)[Neu5Ac(α 2-6)][Fuc(α 1- |
| | 4)]GlcNAc(β1-)B- |
| Disialyl Lewis c | Neu5Ac(α 2-3)Gal(β1-3)[Neu5Ac(α 2-6)]GlcNAc(β1-)B- |
| F1 Alpha | Gal(b1-4)GlcNAc(b1-6)GalNAc(α 1-)Ser/Thr-B- |
| fucosyl GM1 | (Fucal-2Galβ1-3GalNAcβ1-4[NeuAca2-3]-Galβ1- |
| | 4Glcβ1-B- |
| GA1: (Gg4Cer) | Galβ1,3GalNAcβ1,4Galβ1,4Glcβ1-B- |
| GA2: (Gg3Cer) | GalNAcβ1,4Galβ1,4Glcβ1-B- |
| Gal α 1-3Galβ1-4GlacNAc | Gal α 1-3Galβ1-4GlacNAc-B- |
| Galili, Gala-3′LacNAc | Galα1-3Galβ1-4GlcNAcβ-B- |
| Gala | Gal α 1,4Galβ-B- |
| GalCer: | Galβ1-B- |
| GalNAc-GD1a: | GalNAcβ1,4Galβ1,3GalNAcβ1,4(Neu5Acα2,3)Galβ1,4Glcβ1- |
| (IV3Neu5AcII3Neu5AcGg5Cer) | B- |
| Ganglio | Galβ1,3GalNAcβ1,4Galβ1,4Glcβ-B- |
| GbOse3Cer: (Gb3Cer) | Galα1,4Galβ1,4Glcβ1-B- |
| GbOse4Cer | GalNAcβ1,3Galα1,4Galβ1,4Glcβ1-B- |
| GD1a: | Neu5Acα2,3Galβ1,3GalNAcβ1,4(Neu5Acα2,3)Galβ1,4Glcβ1- |
| (IV3Neu5AcII3Neu5AcGg4Cer) | B- |
| GD1b: (II3(Neu5Ac)2Gg4Cer) | Galβ1,3GalNAcβ1,4(Neu5Acα2,8Neu5Acα2,3)Galβ1,4Glcβ1- |
| | B- |
| GD1b-lactone: | II3[Neu5Ac-(2-8,1-9)-Neu5Ac]Gg4-B- |
| GD1c: (IV3(Neu5Ac)2Gg4Cer) | Neu5Acα2,8Neu5Acα2,3Galβ1,3GalNAcβ1,4Galβ1,4Glcβ1- |
| | B- |
| GD1α: | Neu5Ac α2,3Galβ1,3(Neu5Acα2,6)GalNAcβ1- |
| (IV3Neu5AcIII6Neu5AcGg4Cer) | B-,4Galβ1,4Glcβ1-B- |
| GD2: (II3(Neu5Ac)2Gg3Cer) | GalNAcβ1,4(Neu5Acα2,8Neu5Acα2,3)Galβ1,4Glcβ1- |
| | B- |
| GD3: (II3(Neu5Ac)2LacCer) | Neu5Acα2,8Neu5Acα2,3Galβ1,4Glcβ1-B- |
| GGal | Neu5Ac(α 2-3)DGal(β1-1)-B- |
| GlcCer: | Glcβ1-B- |
| Globo | GalNAcβ1,3Gala1,4Galβ1,4Glcβ-B- |
| Globo-H | Fuc α 2Galβ3GalNAcβ3Gal α 4Galβ4Glcβ1-B- |
| Gb5 | Gal(β1-3)GalNAc(β 1-3)Gal(α1-4)Gal(β 1-4)Glc-B- |
| Gb5 | Galβ3GalNAcβ3Galα4Galβ4Glcβ1-B- |
| monosialyl-Gb5 | SAα3Galβ3GalNAcβ3Galα4Galβ4Glcβ1-B- |
| disialyl-Gb5 | SAα3Galβ3GalNAcβ3(SAa2-3)Galα4Galβ4Glcβ1-B- |
| iso-Gb3 | Galα3Galβ4Glcβ1-B- |
| iso-Gb4 | GalNAcβ3Galα3Galβ4Glcβ1-B- |
| Forssman | GalNAcα3GalNAcβ3Galα4Galβ4Glcβ1-B- |
| GM1a: r (II3Neu5AcGg4Cer) | Galβ1,3GalNAcβ1,4(Neu5Acα2,3)Galβ1,4Glcβ1-B- |
| GM1b: (IV3Neu5AcGg4Cer) | Neu5Acα2,3Galβ1,3GalNAcβ1,4Galβ1,4Glcβ1-B- |
| GM2: (II3Neu5AcGg3Cer) | GalNAcβ1,4(Neu5Acα2,3)Galβ1,4Glcβ1-B- |
| GM2b | Neu5Ac(α 2-8)Neu5Ac(α 2-3)DGal(β1-4)DGlc(β1-1)-B- |
| GM3: (II3Neu5AcLacCer) | Neu5Acα2,3Galβ1,4Glcβ1-B- |
| GM4: (I3Neu5AcαGalCer) | Neu5Acα2-3Galβ1-B- |
| GP1c: | Neu5Acα2,8Neu5Acα2,3Galβ1, |
| (IV3(Neu5Ac)2II3(Neu5Ac)3Gg4Cer) | 3GalNAcβ1,4(Neu5Acα2,8Neu5Acα2,8Neu5Acα2,3)Galβ1, |
| | 4Glcβ1-B- |
| GP1cα: (IV3Neu5AcIII6Neu5Ac, | Neu5Ac α2,3Galβ1,3(Neu5Acα2,6)GalNAcβ1, |
| II3(Neu5Ac)3Gg4Cer) | 4(Neu5Acα2,8Neu5Acα2,8Neu5Acα2,3)Galβ1,4Glcβ1- |
| | B- |
| GQ1b: | Neu5Acα2,8Neu5Acα2,3Galβ1,3GalNAcβ1, |
| (IV3(Neu5Ac)2II3(Neu5Ac)2Gg4Cer) | 4(Neu5Acα2,8Neu5Acα2,3)Galβ1,4Glcβ1-B- |
| GQ1bα: | Neu5Ac |
| (IV3(Neu5Ac)2III6(Neu5Ac)2Gg4Cer) | α2,3Galβ1,3(Neu5Acα2,6)GalNAcβ1,4(Neu5Acα2, |
| | 8Neu5Acα2,3)Galβ1,4Glcβ1-B- |
| GQ1c: | Neu5Acα2,3Galβ1,3GalNAcβ1, |
| (IV3Neu5AcII3(Neu5Ac)3Gg4Cer) | 4(Neu5Acα2,8Neu5Acα2,8Neu5Acα2,3)Galβ1,4Glcβ1- |
| | B- |
| GT1, GT1b | Neu5Ac(α 2-3)DGal(β1-3)DGalNAc(β1-4)[Neu5Ac(α 2- |
| | 8)Neu5Ac(α 2-3)]DGal(β1-4)DGlc(β1-1)-B- |
| GT1a: N(V3(Neu5Ac)2II3Neu5AcGg4Cer) | Neu5Acα2,8Neu5Acα2,3Galβ1,3GalNAcβ1,4(Neu5Acα2, |
| | 3)Galβ1,4Glcβ1-B- |
| GT1b: | Neu5Acα2,3Galβ1,3GalNAcβ1,4(Neu5Acα2,8Neu5Acα2, |
| (IV3Neu5AcII3(Neu5Ac)2Gg4Cer) | 3)Galβ1,4Glcβ1-B- |
| GT1c: (II3(Neu5Ac)3Gg4Cer) | Galβ1,3GalNAcβ1,4(Neu5Acα2,8Neu5Acα2, |
| | 8Neu5Acα2,3)Galβ1,4Glcβ1-B- |
| GT1α: | Neu5Acα2,3Galβ1,3(Neu5Acα2,6)GalNAcβ1, |
| (IV3Neu5AcIII6(Neu5Ac)2Gg4Cer) | 4(Neu5Acα2,3)Galβ1,4Glcβ1-B- |
| GT2: | GalNAcβ1,4(Neu5Acα2,8Neu5Acα2, |
| | 8Neu5Acα2,3)Galβ1,4Glcβ1-B- |
| GT3: (II3(NeuAc)3LacCer) | Neu5Acα2,8Neu5Acα2,8Neu5Acα2,3Galβ1,4Glcβ1-B- |
| Internal Lewis x | Gal(β1-4)GlcNAc(β1-3)Gal(β1-4)[Fuc(a1-3)]GlcNAc(β1- |
| | 3)Gal(β1-4)GlcNAc(b1-3)Gal(b1-4)GlcNAc(β1-3)Gal(β1- |
| | 4)Glc(β1-1)-B- |
| Isoglobo | GalNAcβ1,3Gala1,3Galβ1,4Glcβ-B- |
| LacCer: | Galβ1,4Glcβ1-B- |
| Lacto | Galβ1,3GlcNAcβ1,3Galβ1,4Glcβ-B- |
| Lewis a, | Gal(β1-3)[Fuc(α 1-4)]GlcNAc(β1-)B- |
| Lewis b, | Fuc(α 1-2)Gal(β1-3)[Fuc(α 1-4)]GlcNAc(β1-)B- |
| Lewis x, H, | Gal(β1-4)[Fuc(α 1-3)]GlcNAc(β1-)B- |
| Lewis y, | Fuc(α 1-2)Gal(β1-4)[Fuc(α 1-3)]GlcNAc(β1-)B- |
| Mollu | Fuc α 1,4GlcNAcβ1,2Man α 1,3Manβ 1,4Glcβ-B- |
| Muco | Galβ1,3Galβ1,4Galβ1,4Glcβ-B- |
| N-Acetyl GD3 | Neu5Ac(α 2-8)Neu5Ac(α 2-3)Gal(β1-4)Glc(β1-1)-B- |
| Neogala | Galβ 1,6Galβ 1,6Galβ-B- |
| Neolacto | Galβ1,4GlcNAcβ1,3Galβ1,4Glcβ-B- |
| Neu5Ac(a2-3)Gal(b1) | Neu5Ac(α 2-3)Gal(β1-)B- |
| Neu5Ac(a2-3)Gal(b1-3)GalNAc | Neu5Ac(α 2-3)Gal(β1-3)GalNAc-B- |
| Neu5Ac(a2-8)Neu5Ac(a2-3)Gal | Neu5Ac(α 2-8)Neu5Ac(α 2-3)Gal-B- |
| (Sia)₃ | Neu5Acα2-8Neu5Acα2-8Neu5Acα-B- |
| 3′-SL | Neu5Acα2-3Galβ1-4Glcβ-B- |
| N-Glycolyl GM3 | Neu5Gc(α 2-3)Gal(β1-4)Glc(β1-1)-B- |
| NOR1 | Gal(α 1-4)GalNAc(β 1-3)Gal(α a 1-4)Gal(β 1-4)Glc- |
| | B- |
| NOR2 | Gal(α 1-4)GalNAc-(β 1-3)Gal(α 1-4)GalNAc(β 1- |
| | 3)Gal(α 1-4)Gal(β 1-4)Glc-B- |
| NOT int | GalNAc(β 1-3)Gal(α 1-4)GalNAc(β1-3)Gal-(α 1- |
| | 4)Gal(β 1-4)Glc-B- |
| O antigen | Fuc (α 1-2)Gal(β1-3)GlcNAc(β1-3)Gal-B-, |
| OAc-GT1b | Neu5Ac(α 2-3)DGal(β1-3)DGalNAc(β1- |
| | 4)XNeu5Ac9Ac(α 2-8)Neu5Ac(α 2-3)]DGal(β- |
| | 4)DGlc(β1-1)-B- |
| P antigen (Gb4) | Gal(α 1-4)Gal-(β 1-4)GlcNAc(β 1-3)Gal(β 1-4)Glc- |
| | B- |
| Pk Antigen (Gb3) | Gal(α 1-4)Gal(β1-4)Glc-B- |
| Pi | Galα1-4Galβ1-4GlcNAcβ-B- |
| Schisto | GalNAcβ1,4Glcβ-B- |
| Sialyl Lewis a, | Neu5Ac(α 2-3)Gal(β1-3)[Fuc(α 1-4)]GlcNAc(β1-)B- |
| Sialyl Lewis c | Neu5Ac(α 2-3)Gal(β1-3)GlcNAc(β1-)B- |
| Sialyl Lewis x, | Neu5Ac(α 2-3)Gal(β1-4)[Fuc(α 1-3)]GlcNAc(β1- |
| | 3)Gal(β1-4)GlcNAc(β1-3)Gal(β1-)B- |
| Sialyl Lewis x, | Neu5Ac(α 2-3)Gal(β1-4)[Fuc(α 1-3)]GlcNAc(β1-)B- |
| Sialyl Lewis x-i | Neu5Ac(α 2-3)Gal(β1-4)[Fuc(α 1-3)]GlcNAc(β1- |
| | 3)Gal(β1-4)GlcNAc(β1-3)Gal(β1-)B- |
| LNT | Galβ1-3GlcNAcβ1-3Galβ1-4Glcβ-B- |
| LNnT | Galβ1-4GlcNAcβ1-3Galβ1-4Glcβ-B- |
| Sialyl-TF | Neu5Ac(α 2-6) Gal (β1-3) α GalNAc-B-, |
| sialyl-Tn | Neu5Ac(α 2-6)GalNAc-B-, |
| sLac | NeuAc-Gal0-3GlcNAcB-3GalB-4Glc-B- |
| Spirometo | Galβ1,4Glcβ1,3Galβ-B- |
| Sulfatide: | Sulfate3Galβ1-B- |
| TF/Core-1 | α Gal (β1-3)aGalNAc-B-, |
| 3′-sialyl-TF | Neu5Acα2-3Galβ1-3GalNAcα-B- |
| Tn | α GalNAc-B- |
| 6-SiaβTF | Neu5Acβ2-6(Galβ1-3)GalNAcα-B- |
| 3-LacNAc-Tₙ | Galβ1-4GlcNAβ1-3GalNAcα-B- |
| 6-LacNAc-Tₙ | Galβ1-4GlcNAβ1-6GalNAcα-B- |
| 6′SLN | Neu5Acα2-6Galβ1-4GlcNAcβ-B- |
| Core 2 | GlcNAcβ1-6(Galβ1-3)GalNAcα-B- |
| Core 4 | GlcNAcβ1-3(GlcNAcβ1-6)GalNAcα-B- |
| Trifucosyl-Lewis b Antigen | Fuc(α 1-2)Gal(β1-3)[Fuc(α 1-4)]GlcNAc(b1-3)Gal(β1- |
| | 3)[Fuc(α 1-4)]GlcNAc(β1-)B- |
| Trifucosyl-Lewis y Antigen | Fuc(α 1-2)Gal(β1-4)[Fuc(α 1-3)]GlcNAc(b1-3)Gal(β1- |
| | 4)[Fuc(α 1-3)]GlcNAc(β1-)B- |
| Type 1 | GalNAc(α 1-3)[Fuc(α 1-2)]Gal(β1-3)GlcNAc[Fuc(α 1-4)]- |
| | B-, |
| Type 1 A | GalNAc(α1-3)[Fuc(α 1-2)]Gal(β1-3)GlcNAc(β1-3)Gal-B-, |
| Type 2 A | GalNAc(α1-3)[Fuc(α 1-2)]Gal(β1-4)GlcNAc-B-, |
| Type 3 A | GalNAc(α1-3)[Fuc(α 1-2)]Gal(β1-3)GalNAc(β1- |
| | 3)Gal[Fucα 1-2)-B-, |
| Type 4 A | GalNAc(α1-3)[Fuc(α 1-2)]Gal(β1-3)GalNAc(β1-3)Gal(a |
| | 1-4)Gal(β 1-4)Glc-B-, |
| VIM-2 | Neu5Ac(α 2-3)Gal(β1-4)GlcNAc(β1-3)Gal(β1-4)[Fuc(α- |
| | 3)]GlcNAc(β1-)B- |
| Type 4 A | GalNAc(α1-3)[Fuc(α 1-2)]Gal(β1-3)GalNAc(β1-3)Gal(a |
| | 1-4)Gal(β 1-4)Glc-B-, |
| Man₃ | Manα1-3(Manα1-6)Manα-B- |
| 3′SLN | Neu5Acα2-3Galβ1-4GlcNAcβ-B- |
| 6′SL | Neu5Acα2-6Galβ1-4Glcβ-B- |

B is a linker or absent.

Optionally R₃ may be a polysaccharide which comprises two or more polysaccharides selected from i) to v) connected to each other.

X is O or NH.

Preferably the polysaccharide (P2) comprises 3 to 100 monosaccharide moieties, more preferably comprising 2 to 30 monosaccharide moieties.

Preferably the peptide comprises 2 to 100 amino acids, more preferably 2 to 30, most preferably 2 to 20 amino acids.

Optionally the heterologous lipid carrier may be further modified with at least one further group A, wherein A is selected from phosphoryl, sulfate, acetyl, TF disaccharide, Core-1 structure, Tn monosaccharide, Sialyl-TF mono- or disialylated, Sialyl-Tn, Polysialic acid, or a mannose-6-phosphate moiety.

In a further embodiment, the heterologous lipid carrier is Monosialotetrahexosylganglioside (GM1),
wherein in formula (I),

R₁ is —(C₁₆)alkyl;

R₂ is —CH═CH(CH₂)ᵣCH₃;

R₃ is βDGal(1-3) βDGalNAc(1-4)[αNeu5Ac(2-3)] βDGal(1-4) βDGlc(1)-;
r is 12.

In a further embodiment, the heterologous lipid carrier is Monosialotetrahexosylganglioside red (GM1red), wherein in formula (I),
R₁ is —(C₁₆)alkyl;
R₂ is —CH═CHCH₂OH;
R₃ is βDGal(1-3) βDGalNAc(1-4)[αNeu5Ac(2-3)] βDGal(1-4) βDGlc(1)-.

In a further embodiment, the heterologous lipid carrier is Globotriaosylceramide (Gb3), wherein in formula (I),
R₁ is —(C₁₇)alkyl
R₂ is —CH═CH(CH₂)ᵣCH₃
r is 12
R₃ is αD-Gal(1-4) βDGal(1-4) βD-Glc-.

In a further embodiment, the heterologous lipid carrier is a GM1-Gb3 chimera having the formula (III)

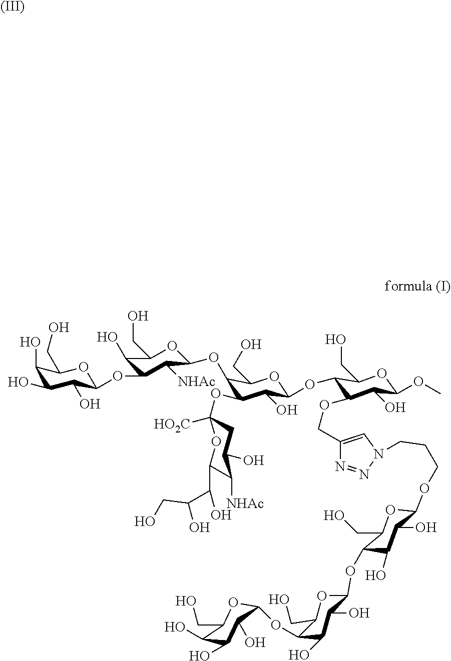

wherein in formula (I)
R₁ is —(C₁₆)alkyl or —(C₁₇)alkyl;
R₂ is —CH═CHCH₂OH or —CH═CH(CH₂)ᵣCH₃;
r is 12
R₃ is the non-lipid part of formula (III).

In a further embodiment, the heterologous lipid carrier is Ganglioside GD1a, wherein in formula (I),
R₁ is —(C₁₆)alkyl;
R₂ is —CH═CH(CH₂)ᵣCH₃;
r is 12;
R₃ is αNeu5Ac(2-3)βDGal(1-3)βDGalNAc(1-4)[αNeu5Ac(2-3)]βDGal(1-4)βDGlc(1)-.

In further embodiment, the heterologous lipid carrier is a Ganglioside selected from the group consisting of asialo-GM1, asialo-GM2, GM2, GM3, GM4, GD2, GD3, GD1b, GT1b, GT1c, GT3, GQ1c, GA1, GA2, GM1b, GalCer, GalNAc-GD1a, GbOse3Cer, GbOse4Cer, GD1b-lactone, GD1c, GD1a, GGal, GlcCer, Globo, Globo-H, Gb5, monosialyl-Gb5, disialyl-Gb5, iso-Gb3, iso-Gb4, Forssman, GM1a, GM1b, GM2b, GP1c, GP1cα, GQ1b, GQ1bα, GT1a, GT1α, GT2, Internal Lewis x, Isoglobo, LacCer, Lacto, Lewis a, Lewis b, Lewis x, Lewis y, Mollu, Muco, N-Acetyl GD3, Neogala, Neolacto, N-Glycolyl GM3, 3′-SL, NOR1, NOR2, OAc-GT1b, Gb4, Gb3, Schisto, Sialyl Lewis a, Sialyl Lewis c, Sialyl Lewis x, Sialyl Lewis x-i, LNT, LNnT, Sialyl-TF, sialyl-Tn, sLac, Spirometo, Sulfatide, TF/Core-1, 3′-sialyl-TF, 3′-sialyl-TF, Tn, 6-SiaβTF, 3-LacNAc-Tₙ, 6-LacNAc-Tₙ, 6′SLN, Core 2, Core 4, Trifucosyl-Lewis b Antigen, Trifucosyl-Lewis y Antigen, Type 1, Type 1 A, Type 2 A, Type 3 A, Type 4 A, VIM-2, 3′SLN, 6′SL.

Preferably, at least 80% heterologous lipid carrier associated with the polysaccharide (P1) remains associated after a treatment with 0.3% bile salts, optionally in combination with pancreatine juice in PBS or DPBS for at least 1 hour at least 37° C.

Further, the invention is directed to a method for preparing a polysaccharide complex, comprising the step of
a) combining the polysaccharide (P1) and a heterologous lipid carrier as defined in claims 1 to 4 in a solution.

Preferably, the polysaccharide (P1) is autoclaved before combining with the heterologous lipid carrier at a temperature of 80 to 150° C., more preferably at 95 to 140° C., most preferably at 110 to 130° C.

Preferably, the polyssacharide (P1) and the heterologous lipid carrier are combined at a temperature of at least 37° C., more preferably 50° C., most preferably of at least 60° C., even more preferably at a temperature of at least 65° C., particularly preferably of at least 70° C. for 2 to 8 h, preferably for 2.5 to 6 h, more preferably for 2.8 to 4 h.

Preferably, the polysaccharide (P1) and the heterologous lipid carrier are combined at a temperature of at least 26 to 45° C., preferably of at least 30 to 40° C., more preferably 35 to 40° C. for at least 10 h, preferably for at least 12 h.

Furthermore, the invention is directed to a polysaccharide complex preparable by the method as described above.

The invention is further directed to a composition comprising
i) one or more of the polysaccharide complex(es) as described above; preferably said composition comprises a mixture of same or different polysaccharide complex(es) as described above and/or
ii) one or more of the polysaccharide complex(es) as described above and at least one pharmaceutically acceptable carrier.

“Pharmaceutically acceptable carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water is a preferred carrier when the pharmaceutical composition is administered orally. Saline and aqueous dextrose are preferred carriers when the pharmaceutical composition is administered intravenously. Saline solutions and aqueous dextrose and glycerol solutions are preferably employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsions, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E.W. Martin. Such compositions will contain a therapeutically effective amount of the therapeutic, preferably in purified form, together with a suitable amount of carrier so as to provide the form for proper administration to the patient. The formulation should suit the mode of administration.

The invention includes the polysaccharide complex of claims or the composition as described above for use in medicine.

The invention includes the polysaccharide complex of claims or the composition as described above for use in the treatment of a subject comprising
i) binding and/or reducing toxicity of and/or neutralizing a toxin;
ii) binding and/or reducing the pathogenicity and/or neutralizing a pathogenic microorganism;
iii) binding a receptor of a toxin;
iv) binding a receptor of a pathogenic microorganism;
v) eliciting or modulating an immune response;
vi) preventing disease
vii) monitoring the development of a disease and/or assessing the efficacy of a therapy of a disease;
viii) delivering a pharmaceutically active compound, preferably said delivering is to a mucosal tissue of a subject.

The invention is further directed to the polysaccharide complex of claims or the composition as described above for use in an in vivo or in vitro method of diagnosis.

The composition is suitable for oral, enteral, dermal, topical, urogenital, inhalational administration.

The composition may be used as nutraceutical.

The invention further comprises a vaccine or adjuvant comprising the polysaccharide complex or composition as described above. The vaccine or adjuvant is suitable for oral or enteral administration.

Better understanding of the present invention and of its advantages will be had from the following examples, offered for illustrative purposes only. The examples are not intended to limit the scope of the present invention in any way.

### Examples of the Invention

### Material & Methods

### Glycolipids:

Monosialoganglioside GM1 is a glycosphingolipid composed of a ceramide (sphingosine and fatty Acid) and an oligosaccharide. Monosialotetrahexosylganglioside-red (GM1-red; reductive Ozonized GM1) is a GM1-structure without sphingosine. The oligosaccharide part of both structures is known to bind the cholera toxin (CT).

Globotriaosylceramide (Gb3) is a glycosphingolipid. Its oligosaccharide part is known to be a natural receptor for the shiga toxins (Stxs).

### Loading Protocol.

Corn Starch was resuspended in PBS (i.e. at 5 g/l) and autoclaved for 20 minutes at 121° C. At the end of the autoclaving, the starch solution was kept at i.e. 70° C. under stirring conditions and 1 to 5 μg/ml of glycolipid (i.e. GM1, Gb3 or GM1-red) previously resuspended in DMF or ethanol was added. The solution was incubated for 3 hours at i.e. 70° C. under stirring condition. Alternatively, the starch solution was added with 1 to 5 μg/ml glycolipid and incubated overnight at i.e. 37° C. under stirring condition. The starch was then centrifuged and washed 3 times with PBS or PBS-Twenn 0.05% to remove free GM1-glycolipids, before being resuspended in PBS or DPBS and stored at RT or 4° C.

### Labeling of Toxin.

For ELISA, toxins (CT ad Stx1) were labelled with Alkaline Phosphatase (AP) according to the manufacturer instructions using Lightning-Link® Alkaline Phosphatase Antibody Labeling Kit (Novus Biologicals Europe/UK, Abingdon, United Kingdom Catalog Number in May 2019: 702-0030).

For fluorescence microscopy, toxins (CT ad Stx1) were labelled with Texas Red) according to the manufacturer instructions using the conjugation kit (fast)-Lightning-Link (abcam). The labeled CT-Texas Red (CT-Tx) and Stx1-Texas Red (Stx1-Tx) were stored at 4° C.

### ELISA.

The Starch loaded with a glycolipid was diluted in PBS containing 2% FKS or BSA and incubated at Room temperature 37° C. for 1 hour to prevent unspecific binding. The starch was than washed, re-suspended in PBS containing 1% FKS/BSA, added with the AP-labelled CT or STx and incubated for 1 to 2 hours at 37° C.

The Starch was washed 3 times with PBS or PBS+0.02% Tween 20 and resuspended in PBS. 50 μl of the starch suspensions were given in triplicate on an ELISA plate. Two wells were inoculated with a suitable substrate (for AP), the reactions were stopped, and extinction measured at a suitable wavelength (e.g., 580 nm for AP). The third well was used to determine the OD in the final cell suspension and the extinction measured previously and standardized to OD1. Standardized results allowed to compare the binding strength of different strains and/or preparations.

### Fluorescence Microscopy

200 μl of a glycolipid-loaded Starch were washed, resuspended in 2% FKS-PBS (for CT) or 2% BSA-PBS (for Stx1) and incubated at 37° C. for 45 min to avoid unspecific binding. Afterwards the suspensions were washed and the pellets resuspended in 200 μl of diluted CT-Tx in 1% FKS-PBS or Stx1-Tx 1% BSA-PBS and incubated at 37° C. for 90 min. Pellets were washed extensively with PBS and resuspended in 200 μl PBS.

20 μl of each suspension was dropped on a microscope slide and incubated for 20 min at room temperature in the dark until the drops were well dried. 10 μl of ROTI®Mount FluorCare (Mounting Media for Fluorescence Microscopy; Carl Roth) was dropped and covered with a cover slip. The microscopic observation was performed using fluorescent microscope Keyence BZ-9000. The Texas red was detected at 596/615 exct/Emis.

### Example 1: GM1 Loading

To test the ability of starch to stably associate the glycolipid GM1 and to present the oligosaccharide moiety to the environment (e.g., on the exterior), said starch was loaded with GM1 and the ability to bind AP-cholera toxin was tested by the means of ELISA.

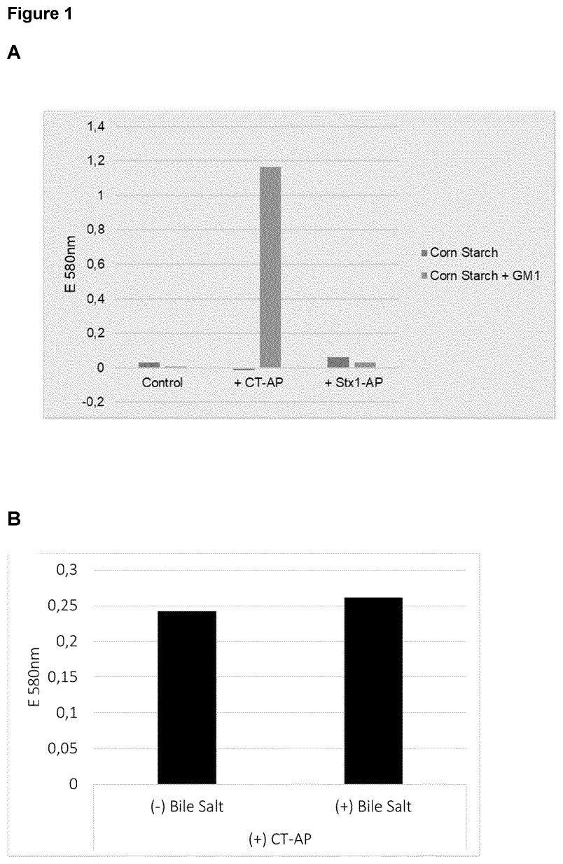

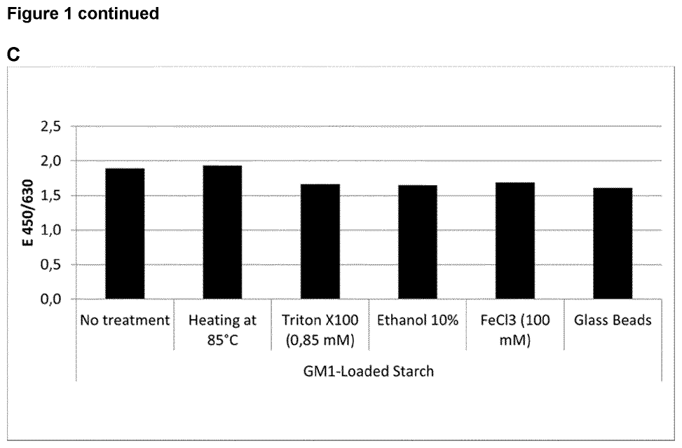

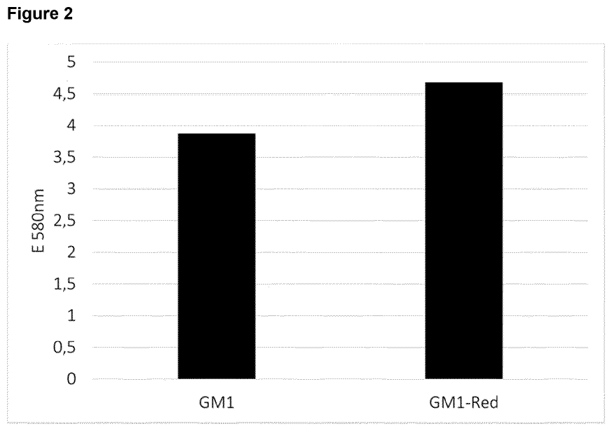

As presented in FIG. 1a, no signal was observed for unloaded corn starch (treated exactly as the corn starch that was loaded) in presence of the substrate used to identify the presence of AP (control). Also, no signal could be observed when unloaded starch was incubated with AP-labelled CT (+CT-AP) or AP-labelled Stx1 (Stx1-AP) prior to addition of the substrate. These results demonstrated that corn starch does not spontaneously interact with CT, Stx1 or AP.

The corn starch loaded with GM1 also did not present any background-signal when incubated with the substrate used to identify the presence of AP. Furthermore, no signal cloud be observed when it was incubated with the AP-labelled shiga toxin 1 (Stx-1) prior to addition of the AP-substrate. This demonstrated that the loading with GM1 did not result in binding of the AP. In contrast, when incubated with CT-AP corn starch loaded with GM1 presented a strong signal, confirming that the GM1-loaded corn starch presented a strong and specific CT-binding.

To test the stability of the association between GM1 and corn starch, the GM1-loaded starch was subjected to treatment with 0.3% bile salt for 1 hour at 37° C. before being tested for its ability to bind labelled CT. As presented in FIG. 1b, the treatment of GM1-loaded corn starch with bile salt did not result in a reduction of the signal, demonstrating that the association is stable under physiological conditions encountered in the gut.

To further assess the stability of the association between GM1 and corn starch, the GM1-loaded starch was subjected to several treatments that were reported to strongly damage the integrity of membrane lipid bilayer systems (ref).

The GM1 loaded starch was submitted to:
i) Treatment with Triton X-100 at final concentrations of 0.85 mM at room temperature for 15 min.
ii)Treatment with ethanol 10% at room temperature for 15 min.
iii) Treatment with FeCl₃ at a final concentration of 100 mM at room temperature for 15 min.
iv) Vortex with glass beads at 3000 rpm for 4 min.
v) Heating at 85° C. for 15 min.

At the end of the treatment, the starch was washed 3 times with PBS to remove potentially released GM1. The GM1-loaded starch was than blocked with PBS containing 2% FBS followed by incubation with HRP-labelled CT-subunit B for 2 hours at 37° C.

The Starch was washed 3 times with PBS or PBS and resuspended in PBS. 50 μl of the starch suspensions were given in triplicate on an ELISA plate. Two wells were inoculated with a suitable substrate (for HRP), the reactions were stopped, and extinction measured at a suitable wavelength (e.g., 450 vs 630). The third well was used to determine the OD in the final suspension and to standardize the results.

As presented and FIG. 1c none of the treatment resulted in a significant reduction of the signal, demonstrating that the association is stable under conditions that resulted in damaging membrane lipid bilayer system like liposomes etc.

Similarly, further conditions have been tested to explore the stability of the polysaccharide complex:

TABLE 2
Stability of the polysaccharide complexes

| Conditions | with GM1 |
|---|---|
| pH 1, 15 min RT | (−) |
| pH 9, 15 min RT | (−) |
| 75° C. 15 min | (−) |
| Ultrasonic bath 2 min RT | (−) |

### Example 2: Loading of GM1-red

To test the role of the sphingosine part of the glycolipid GM1 in the association with the starch, said starch was loaded with GM1 and GM1-red, and the ability to bind AP-cholera toxin was tested by the means of ELISA.

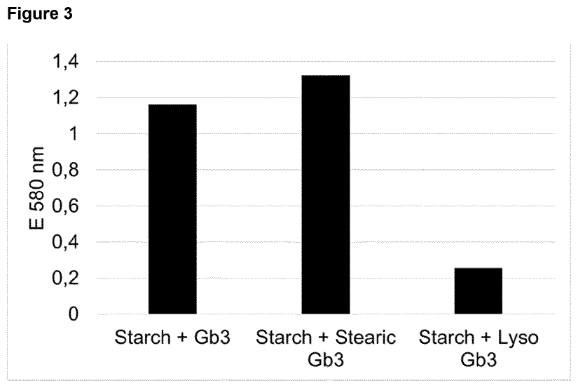

As presented in FIG. 2, corn starch loaded with GM1 and GM1-red presented similar signal after incubation with CT-AP. The absence of sphingosine did not result in a reduction of the loading efficacy, suggesting that the sphingosine may not be necessary for the association.

### Example 3: Loading of Gb3

### Gb3 Binding

To test the ability of starch to associate another glycolipid and to assay the role of the fatty acid part of the glycolipid in the association with starch, said starch was loaded with different Gb3 and the ability to bind AP-Stx1 was tested by the means of ELISA.

Thus, starch was loaded with isolated Gb3, stearic-Gb3 and Lyso-Gb3. Gb3 isolated from animals is composed of a mix of Gb3 containing fatty acids of different length, mostly between 20 and 24 carbons. Stearic Gb3 is a pure population containing only stearic acid as fatty acid. Lyso-Gb3 is a GB3-structure that is missing the fatty acid part.

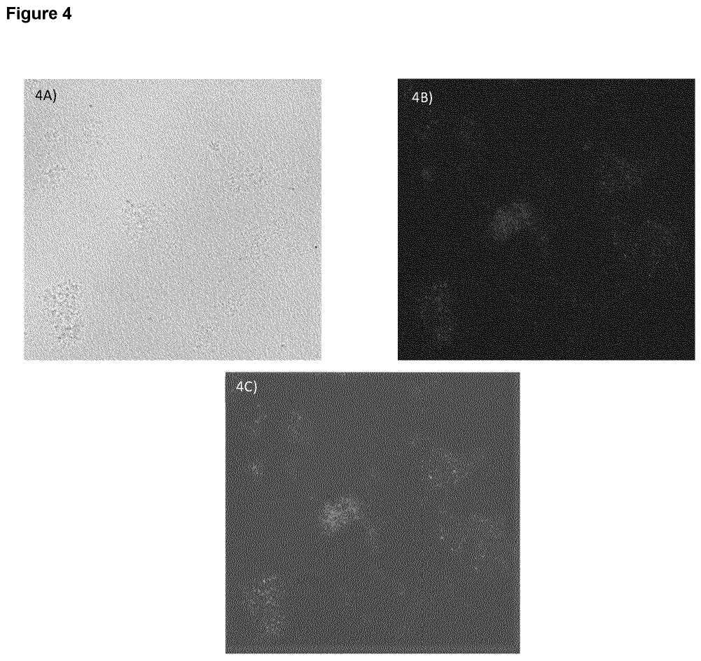

As presented in FIG. 3, Corn starch loaded with Gb3 and stearic-Gb3 presented a strong signal after incubation with Stx1-AP. In contrast, corn starch loaded with lyso-Gb3 presented a very weak signal. These results demonstrated that com-starch is able to associate Gb3 containing fatty acids of different lengths and that this fatty acid part is essential for the association.

### Example 4: Fluorescence Microscopy

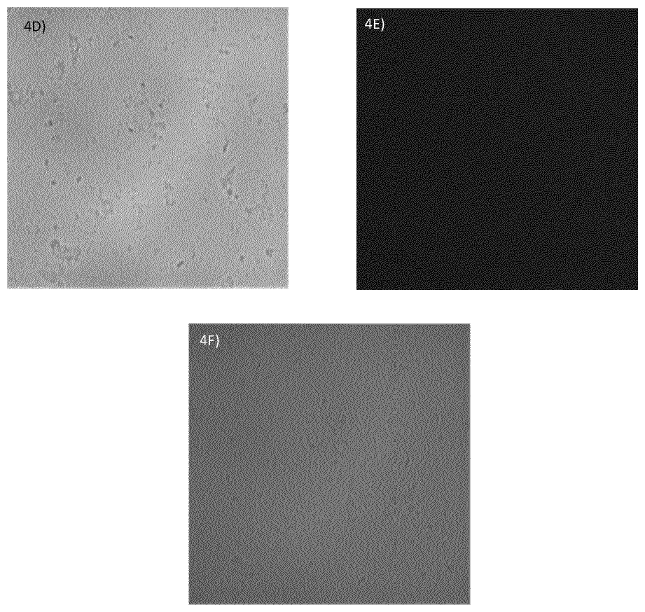

As presented in FIG. 4, no signal could be detected for GM1-loaded Starch incubated with Stx1-Tx (FIG. 4F), demonstrating that GM1-loaded starch does not unspecific ally bind Texas-red or an unrelated protein (Stx1). In contrast, GM1-loaded starch incubated with CT-Tx presented a strong signal (FIG. 4B) that co-localize with the starch (FIGS. 4A and 4C). This result demonstrate that GM1-is associated to the starch and specifically binds CT-Tx.

1. Polysaccharide complex comprising
i) at least one polysaccharide (P1) and
ii) a heterologous lipid carrier wherein the heterologous lipid carrier comprises
a) a lipid portion comprising at least one ceramide-like glycolipid moiety and/or a fatty acid moiety and
b) at least one non-lipid moiety, preferably said non-lipid portion comprises at least one carbohydrate moiety, at least one lipopeptide moiety, at least one linker, at least one chemical compound, and/or at least one peptide moiety,
wherein i) and ii) are associated by direct bonding to each other.

2. The polysaccharide complex of claim 1, wherein
I) i) and ii) are associated by non-covalent bonds and/or
II) the polysaccharide complex, in particular the bonding between i) and ii) is stabile against at least one of the following conditions:
a) treatment with Triton X-100 at final concentrations of 0.85 mM at room temperature for 15 min and/or
b) treatment with ethanol 10% at room temperature for 15 min and/or
c) treatment with FeCl₃ at a final concentration of 100 mM at room temperature for 15 min and/or
d) incubation at pH1 and/or pH9 at room temperature for 15 min and/or
e) vortex with glass beads at 3000 rpm for 4 min and/or
f) incubating at 75 to 85° C. for 15 min and/or
g) treatment with 0.3% bile salt for 1 hour at 37° C.

3. The polysaccharide complex of claim 1 or 2 wherein
the heterologous lipid carrier is defined according to the general formula (I) or (II); 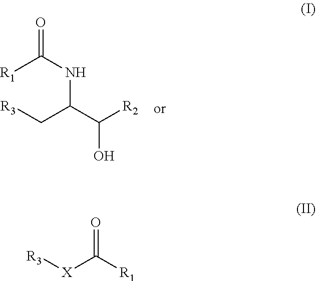
wherein i) is associated with ii);
wherein
R₁ is linear or branched (C₁-C₈₀)alkyl, (C₂-C₈₀)alkenyl; preferably (C₁₀-C₅₀)alkyl, (C₁₀-C₅₀)alkenyl, more preferably (C₁₁-C₃₀)alkyl, (C₁₁-C₃₀)alkenyl, most preferably (C₁₂-C₂₅)alkyl, (C₁₂-C₂₅)alkenyl;
or R₁ is —C(OH)—R₄, wherein R₄ is linear or branched (C₁-C₈₀)alkyl or (C₂-Cao)alkenyl;
or R₁ is (C₆-C₈₀)alkyl or (C₆-C₈₀)alkenyl wherein:
(i) (C₆-C₈₀)alkyl or (C₆-C₈₀)alkenyl is substituted with at least one (C₅-C₁₅)cycloalkyl, (C₆-C₁₅)cycloalkenyl, heterocyclyl, or aromatic ring; or
(ii) (C₆-C₈₀)alkane or (C₆-C₈₀)alkenyl includes, within the (C₆-C₈₀)alkyl or (C₆-C₈₀)alkenyl chain, at least one (C₅-C₁₅) cycloalkyl, (C₅-C₁₅) cycloalkyl, heterocyclyl, or aromatic ring;
R₂ is —CH₂(CH₂)ₙCH₃, —CH(OH)(CH₂)ₒCH₃, —CH(OH)CH(OH)(CH₂)ₚCH₃, —CH═CHCH₂OH, —CH(OH)(CH₂)_{q}CH(CH₃)₂, —CH═CH(CH₂)ᵣCH₃, —CH(OH)(CH₂)ₛCH(CH₃)CH₂CH₃,
n, o, p, q, and r are independently selected from is an integer ranging from 4-17;
R₃ is an α-linked or a β-linked mono, di- or polysaccharide (P2) moiety, at least one peptide moiety, or a polysaccharide peptide moiety comprising an α-linked or a β-linked mono-, di- or polysaccharide and at least one peptide;
optionally R₃ comprises at least one linker in addition to the α-linked or the β-linked mono, di- or polysaccharide (P2) moiety, the at least one peptide moiety, or the polysaccharide peptide moiety comprising an α-linked or a β-linked mono-, di- or polysaccharide and at least one peptide;
preferably the polysaccharide (P2) comprises 3 to 100 monosaccharide moieties, more preferably comprising 2 to 30 monosaccharide moieties;
preferably the peptide comprises 2 to 100 amino acids, more preferably 2 to 30, most preferably 2 to 20 amino acids;
optionally R₃ is selected from the group consisting of: a polysaccharide which may comprise acetate groups, most preferably the first sugar of the said carbohydrate is galactose, a glucose, a mannose, a xylose, a neuraminic acid, a N-acetyl glucosamine, N-acetyl galactosamine or a galacturonic acid;
optionally the heterologous lipid carrier may be further modified with at least one further group A, wherein A is selected from phosphoryl, sulfate, acetyl, TF disaccharide, Core-1 structure, Tn monosaccharide, Sialyl-TF mono- or disialylated, Sialyl-Tn, Polysialic acid, or a mannose-6-phosphate moiety;
X is O or NH.

4. The polysaccharide complex according to claim 3, wherein in formula (I) or (II), R₃ is selected from
i) Galβ1 3GalNAcβ1 4(Neu5Acα2,3)Galβ1 4Glcβ1-B—, or 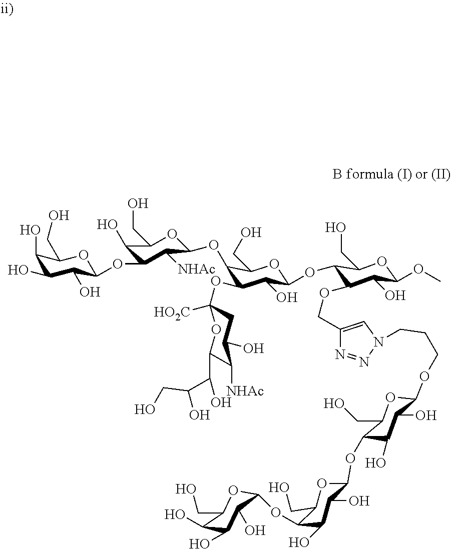
or
iii) αNeu5Ac(2-3) βDGal(1-3) βDGalNAc(1-4)[α Neu5Ac(2-3)]βDGal(1-4) βDGlc(1)-B—;
N-Acetyl-D-galactose-β-1,4-[N-Acetylneuraminidate-α-2,3-]-Galactose-β-1,4-glucose(1)-B—;
βDGalNAc(1-4)[αNeu5Ac(2-8)αNeu5Ac(2-3)]βDGal(1-4)βDGlc(1)-B—; βDGal(1-3) βDGalNAc(1-4)[αNeu5Ac(2-8)αNeu5Ac(2-3)]βDGal(1-4)βDGlc(1)-B—; βDGal(1-3)βDGalNAc(1-4)[αNeu5Ac(2-8)αNeu5Ac(2-3)]βDGal(1-4)βDGlc(1)-B—; αNeu5Ac(2-3) βDGal(1-3)βDGalNAc(1-4)[αNeu5Ac(2-8) αNeu5Ac(2-3)] βDGal(1-4) βDGlc(1)-B—;
βDGal(1-3)βDGalNAc(1-4)[αNeu5Ac(2-8)αNeu5Ac(2-8)αNeu5Ac(2-3)]βDGal(1-4) βDGlc(1)-B-Neu5Acα2,3Galβ1,3GalNAcβ1,4(Neu5Acα2,8Neu5Acα2,8Neu5Acα2,3)Galβ1,4Glcβ1-B—; Galβ1,3GalNAcβ1,4Galβ1,4Glcβ1-B—; Neu5Acα2,3Galβ1,3GalNAcβ1,4Galβ1,4Glcβ1-B—; Neu5Acα2,3Galβ1,4Glcβ1-B—; Neu5Acα2,8Neu5Acα2,3Galβ1,4Glcβ1-B—; Neu5Acα2,8Neu5Acα2,8Neu5Acα2,3Galβ1,4Glcβ1-B—; GalNAc(β1-3)Gal(α1-4)Gal(β1-4)Glc(81)-B—; or
iv) Gal α1-3Galβ1-4GlacNAc-B—, Gal(α1-4)Gal(β1-4)GlcNAc-B—, Gal(α1-4)Gal(β1-4)Glc-B—, NAc-((1-3)Gal(β1-4)Glc-B—, Gal(α1-4)Gal(β1-4)GlcNAc(β1-2) Man-B—; or
v) any of the following glycan moieties listed in column 2, titled with “Structure” of Table 1:
TABLE 1
Glycan moieties

| Column 1: Example | Column 2: Structure |
|---|---|
| 2-6 Sialyl i-Lewis x | Neu5Ac(α 2-6)Gal(β1-4)GlcNAc(β1-3)Gal(β1-4)[Fuc(α |
| | 1-3)]GlcNAc(β1-)B- |
| 3′-Sulfo Lewis a | HSO3(-3)Gal(β1-3)[Fuc(a1-4)]GlcNAc(β1-)B- |
| 3′-Sulfo Lewis x | HSO3(-3)Gal(β1-4)[Fuc(a1-3)]GlcNAc(β1-)B- |
| 6,6′-Disulfo Sialyl Lewis x | Neu5Ac(α 2-3)[HSO3(-6)]Gal(β1-4)[Fuc(α 1- |
| | 3)][HSO3(-6)]GlcNAc(β1-)B- |
| 6-Sulfo Lewis x | Gal(β1-4)[Fuc(α 1-3)][HSO3(-6)]GlcNAc(β1-)B- |
| 6′-Sulfo Sialyl Lewis x | Neu5Ac(α 2-3)[HSO3(-6)]Gal(β1-4)[Fuc(α 1- |
| | 3)]GlcNAc(β1-)B- |
| 6^{(GlcNAc)}-su-SLe^{x} | Neu5Acα2-3Galβ1-4(Fucα1-3)(6-O-Su)GlcNAcβ-B- |
| 6′-Sia-6-Su-LacNAc | Neu5Acα2-6Galβ1-4(6-O-Su)GlcNAcβ-B- |
| 6-Su-3′SiaLe^{c} | Neu5Acα2-3Galβ1-3(6-O-Su)GlcNAcβ-B- |
| 6-Su-3′SLN | Neu5Acα2-3Galβ1-4(6-O-Su)GlcNAcβ-B- |
| 3′SLN(Gc) | Neu5Gcα2-3Galβ1-4GlcNAcβ-B- |
| 6′SLN(Gc) | Neu5Gcα2-6Galβ1-4GlcNAcβ-B- |
| GlcNAcβ3′LacNAc | GlcNAcβ1-3Galβ1-4GlcNAcβ-B- |
| Isomaltotriose | Glcα1-6Glca1-6Glcβ-B- |
| Chitotriose | GlcNAcβ1-4GlcNAβ1-4GlcNAcβ-B- |
| alpha Gal epitope, | Gal(α 1-3)Gal(β1-4)GlcNAc-B- |
| Arthro | GlcNAcβ1,3Manβ1,4Glcβ-B- |
| Aₜᵣᵢ | GalNAcα1-3(Fucα1-2)Galβ-B- |
| Bₜᵣᵢ | Galα1-3(Fucα1-2)Galβ-B- |
| Gal₂3,4-GlcNAc | Galβ1-4(Galβ1-3)GlcNAcβ-B- |
| asialo-GM1, GA1 | DGal(β1-3)DGalNAc(β1-4)DGal(β1-4)DGlc(β1-1)-B- |
| asialo-GM2, GA2 | DGalNAc(β1-4)DGal(β1-4)DGlc(β1-1)-B- |
| Blood Group A Trisaccharide | GalNAc(α 1-3)[Fuc(α 1-2)]Gal(β1-)B- |
| Blood Group A Type 1 | GalNAc(α 1-3)[Fuc(α 1-2)]Gal(β1-3)GlcNAc(β1-)B- |
| Blood Group A Type 1 | GalNAc(α 1-3)[Fuc(α 1-2)]Gal(β1-3)[Fuc(α 1- |
| (difucosyl) | 4)]GlcNAc(β1-)B- |
| Blood Group A Type 2 | GalNAc(α 1-3)[Fuc(α 1-2)]Gal(β1-4)GlcNAc(β1-)B- |
| Blood Group A Type 2 | GalNAc(α 1-3)[Fuc(α 1-2)]Gal(β1-4)[Fuc(α 1)- |
| (difucosyl) | 3)]GlcNAc(β1-)B- |
| Blood Group A Type 3 | GalNAc(α 1-3)[Fuc(α 1-2)]Gal(β1-3)GalNAc(α1-)B- |
| Blood Group A Type 4 | GalNAc(α 1-3)[Fuc(α 1-2)]Gal(β1-3)GalNAc(β1-)B- |
| Blood Group B | Gal(α 1-3)[Fuc (α 1-2)]Gal(β1-3)GlcNAc(β1-3)Gal-B-, |
| Blood Group B Type 2 | Gal(α 1-3)[Fuc(α 1-2)]Gal(β1-4)GlcNAc(β1-)B- |
| Blood Group H Type 1, | Fuc(α 1-2)Gal(β1-3)GlcNAc(β1-)B- |
| Blood Group H Type 2, | Fuc(α 1-2)Gal(β1-4)GlcNAc(β1-)B- |
| Blood Group H Type 3, | Fuc(α 1-2)Gal(β1-3)GalNAc(α1-)B- |
| Blood Group H, | Fuc(α 1-2)Gal(β1-)B- |
| Sia6′H (type 2) | Neu5Acα2-6(Fucα1-2)Galβ1-4GlcNAcβ-B- |
| 6-LacNAc-TF | Galβ1-4GlcNAcβ1-6(Galβ1-3)GalNAcα-B- |
| C-Series Ganglio-sides Oligo- | -Gal(β1-3)GalNAc(β1-4)[Neu5Ac(α 2-8)Neu5Ac(α 2- |
| saccharide/Ganglio-tetraosyl | 8)Neu5Ac(α 2-3)]Gal(β1-4)Glc(β1-1)-B- |
| Core Structure | |
| C-Series Ganglio-sides Oligo- | Neu5Ac(α 2-8)Neu5Ac(α 2-8)Neu5Ac(α 2-3)Gal-B- |
| saccharide/Hemato- or | |
| Ganglio-Type | |
| Cyclic Sialyl 6-Sulfo Lewis x | cyclicNeu5Ac(α 2-3)Gal(b1-4)[Fuc(α 1-3)][HSO3(- |
| | 6)]GlcNAc-B- |
| Dimeric Lewis x | Gal(β1-4)[Fuc(α 1-3)]GlcNAc(β1-3)Gal(b1-4)[Fuc(α 1- |
| | 3)]GlcNAc(β1-)B- |
| Disialyl Lewis a | Neu5Ac(α 2-3)Gal(β1-3)[Neu5Ac(α 2-6)][Fuc(α 1- |
| | 4)]GlcNAc(β1-)B- |
| Disialyl Lewis c | Neu5Ac(α 2-3)Gal(β1-3)[Neu5Ac(α 2-6)]GlcNAc(β1- |
| | )B- |
| F1 Alpha | Gal(b1-4)GlcNAc(b1-6)GalNAc(α 1-)Ser/Thr-B- |
| fucosyl GM1 | (Fucal-2Galβ1-3GalNAcβ1-4[NeuAca2-3]-Galβ1- |
| | 4Glcβ1-B- |
| GA1: (Gg4Cer) | Galβ1,3GalNAcβ1,4Galβ1,4Glcβ1-B- |
| GA2: (Gg3Cer) | GalNAcβ1,4Galβ1,4Glcβ1-B- |
| Gal α 1-3Galβ1-4GlacNAc | Gal α 1-3Galβ1-4GlacNAc-B- |
| Galili, Gala-3′LacNAc | Galα1-3Galβ1-4GlcNAcβ-B- |
| Gala | Gal α 1,4Galβ-B- |
| GalCer: | Galβ1-B- |
| GalNAc-GD1a: | GalNAcβ1,4Galβ1,3GalNAcβ1,4(Neu5Acα2,3)Galβ1, |
| (IV3Neu5AcII3Neu5AcGg5Cer) | 4Glcβ1-B- |
| Ganglio | Galβ1,3GalNAcβ1,4Galβ1,4Glcβ-B- |
| GbOse3Cer: (Gb3Cer) | Galα1,4Galβ1,4Glcβ1-B- |
| GbOse4Cer | GalNAcβ1,3Galα1,4Galβ1,4Glcβ1-B- |
| GD1a: | Neu5Acα2,3Galβ1,3GalNAcβ1, |
| (IV3Neu5AcII3Neu5AcGg4Cer) | 4(Neu5Acα2,3)Galβ1,4Glcβ1-B- |
| GD1b: (II3(Neu5Ac)2Gg4Cer) | Galβ1,3GalNAcβ1, |
| | 4(Neu5Acα2,8Neu5Acα2,3)Galβ1,4Glcβ1-B- |
| GD1b-lactone: | II3[Neu5Ac-(2-8,1-9)-Neu5Ac]Gg4-B- |
| GD1c: (IV3(Neu5Ac)2Gg4Cer) | Neu5Acα2,8Neu5Acα2,3Galβ1, |
| | 3GalNAcβ1,4Galβ1,4Glcβ1-B- |
| GD1α: | Neu5Ac α2,3Galβ1,3(Neu5Acα2,6)GalNAcβ1-B-, |
| (IV3Neu5AcIII6Neu5AcGg4Cer) | 4Galβ1,4Glcβ1-B- |
| GD2: (II3(Neu5Ac)2Gg3Cer) | GalNAcβ1,4(Neu5Acα2,8Neu5Acα2,3)Galβ1,4Glcβ1- |
| | B- |
| GD3: (II3(Neu5Ac)2LacCer) | Neu5Acα2,8Neu5Acα2,3Galβ1,4Glcβ1-B- |
| GGal | Neu5Ac(α 2-3)DGal(β1-1)-B- |
| GlcCer: | Glcβ1-B- |
| Globo | GalNAcβ1,3Gala1,4Galβ1,4Glcβ-B- |
| Globo-H | Fuc α 2Galβ3GalNAcβ3Gal α 4Galβ4Glcβ1-B- |
| Gb5 | Gal(β1-3)GalNAc(β 1-3)Gal(a 1-4)Gal(β 1-4)Glc- |
| | B- |
| Gb5 | Galβ3GalNAcβ3Galα4Galβ4Glcβ1-B- |
| monosialyl-Gb5 | SAα3Galβ3GalNAcβ3Galα4Galβ4Glcβ1-B- |
| disialyl-Gb5 | SAα3Galβ3GalNAcβ3(SAa2-3)Galα4Galβ4Glcβ1-B- |
| iso-Gb3 | Galα3Galβ4Glcβ1-B- |
| iso-Gb4 | GalNAcβ3Galα3Galβ4Glcβ1-B- |
| Forssman | GalNAcα3GalNAcβ3Galα4Galβ4Glcβ1-B- |
| GM1a: r (II3Neu5AcGg4Cer) | Galβ1,3GalNAcβ1,4(Neu5Acα2,3)Galβ1,4Glcβ1-B- |
| GM1b: (IV3Neu5AcGg4Cer) | Neu5Acα2,3Galβ1,3GalNAcβ1,4Galβ1,4Glcβ1-B- |
| GM2: (II3Neu5AcGg3Cer) | GalNAcβ1,4(Neu5Acα2,3)Galβ1,4Glcβ1-B- |
| GM2b | Neu5Ac(α 2-8)Neu5Ac(α 2-3)DGal(β1-4)DGlc(β1-1)- |
| | B- |
| GM3: (II3Neu5AcLacCer) | Neu5Acα2,3Galβ1,4Glcβ1-B- |
| GM4: (I3Neu5AcαGalCer) | Neu5Acα2-3Galβ1-B- |
| GP1c: | Neu5Acα2,8Neu5Acα2,3Galβ1, |
| (IV3(Neu5Ac)2II3(Neu5Ac)3Gg4Cer) | 3GalNAcβ1,4(Neu5Acα2,8Neu5Acα2,8Neu5Acα2, |
| | 3)Galβ1,4Glcβ1-B- |
| GP1cα: (IV3Neu5AcIII6Neu5Ac, | Neu5Ac α2,3Galβ1,3(Neu5Acα2,6)GalNAcβ1, |
| II3(Neu5Ac)3Gg4Cer) | 4(Neu5Acα2,8Neu5Acα2, |
| | 8Neu5Acα2,3)Galβ1,4Glcβ1-B- |
| GQ1b: | Neu5Acα2,8Neu5Acα2,3Galβ1,3GalNAcβ1, |
| (IV3(Neu5Ac)2II3(Neu5Ac)2Gg4Cer) | 4(Neu5Acα2,8Neu5Acα2,3)Galβ1,4Glcβ1-B- |
| GQ1bα: | Neu5Ac |
| (IV3(Neu5Ac)2III6(Neu5Ac)2Gg4Cer) | α2,3Galβ1,3(Neu5Acα2,6)GalNAcβ1,4(Neu5Acα2, |
| | 8Neu5Acα2,3)Galβ1,4Glcβ1-B- |
| GQ1c: | Neu5Acα2,3Galβ1,3GalNAcβ1, |
| (IV3Neu5AcII3(Neu5Ac)3Gg4Cer) | 4(Neu5Acα2,8Neu5Acα2,8Neu5Acα2,3)Galβ1, |
| | 4Glcβ1-B- |
| GT1, GT1b | Neu5Ac(α 2-3)DGal(β1-3)DGalNAc(β1-4)[Neu5Ac(α |
| | 2-8)Neu5Ac(α 2-3)]DGal(β1-4)DGlc(β1-1)-B- |
| GT1a: | Neu5Acα2,8Neu5Acα2,3Galβ1,3GalNAcβ1,4(Neu5Ac |
| N(V3(Neu5Ac)2II3Neu5AcGg4Cer) | α2,3)Galβ1,4Glcβ1-B- |
| GT1b: | Neu5Acα2,3Galβ1,3GalNAcB1,4(Neu5Acα2,8Neu5Ac |
| (IV3Neu5AcII3(Neu5Ac)2Gg4Cer) | α2,3)Galβ1,4Glcβ1-B- |
| GT1c: (II3(Neu5Ac)3Gg4Cer) | Galβ1,3GalNAcβ1,4(Neu5Acα2,8Neu5Acα2, |
| | 8Neu5Acα2,3)Galβ1,4Glcβ1-B- |
| GT1α: | Neu5Acα2,3Galβ1,3(Neu5Acα2,6)GalNAcβ1, |
| (IV3Neu5AcIII6(Neu5Ac)2Gg4Cer) | 4(Neu5Acα2,3)Galβ1,4Glcβ1-B- |
| GT2: | GalNAcβ1,4(Neu5Acα2,8Neu5Acα2, |
| | 8Neu5Acα2,3)Galβ1,4Glcβ1-B- |
| GT3: (II3(NeuAc)3LacCer) | Neu5Acα2,8Neu5Acα2,8Neu5Acα2,3Galβ1,4Glcβ1-B- |
| Internal Lewis x | Gal(β1-4)GlcNAc(β1-3)Gal(β1-4)[Fuc(a1- |
| | 3)]GlcNAc(β1-3)Gal(β1-4)GlcNAc(b1-3)Gal(b1- |
| | 4)GlcNAc(β1-3)Gal(β1-4)Glc(β1-1)-B- |
| Isoglobo | GalNAcβ1,3Gala1,3Galβ1,4Glcβ-B- |
| LacCer: | Galβ1,4Glcβ1-B- |
| Lacto | Galβ1,3GlcNAcβ1,3Galβ1,4Glcβ-B- |
| Lewis a, | Gal(β1-3)[Fuc(α 1-4)]GlcNAc(β1-)B- |
| Lewis b, | Fuc(α 1-2)Gal(β1-3)[Fuc(α 1-4)]GlcNAc(β1-)B- |
| Lewis x, H, | Gal(β1-4)[Fuc(α 1-3)]GlcNAc(β1-)B- |
| Lewis y, | Fuc(α 1-2)Gal(β1-4)[Fuc(α 1-3)]GlcNAc(β1-)B- |
| Mollu | Fuc α 1,4GlcNAcβ1,2Man α 1,3Manβ 1,4Glcβ-B- |
| Muco | Galβ1,3Galβ1,4Galβ1,4Glcβ-B- |
| N-Acetyl GD3 | Neu5Ac(α 2-8)Neu5Ac(α 2-3)Gal(β1-4)Glc(β1-1)-B- |
| Neogala | Galβ 1,6Galβ 1,6Galβ-B- |
| Neolacto | Galβ1,4GlcNAcβ1,3Galβ1,4Glcβ-B- |
| Neu5Ac(a2-3)Gal(b1) | Neu5Ac(α 2-3)Gal(β1-)B- |
| Neu5Ac(a2-3)Gal(b1-3)GalNAc | Neu5Ac(α 2-3)Gal(β1-3)GalNAc-B- |
| Neu5Ac(a2-8)Neu5Ac(a2-3)Gal | Neu5Ac(α 2-8)Neu5Ac(α 2-3)Gal-B- |
| (Sia)₃ | Neu5Acα2-8Neu5Acα2-8Neu5Acα-B- |
| 3′-SL | Neu5Acα2-3Galβ1-4Glcβ-B- |
| N-Glycolyl GM3 | Neu5Gc(α 2-3)Gal(β1-4)Glc(β1-1)-B- |
| NOR1 | Gal(α 1-4)GalNAc(β 1-3)Gal(α a 1-4)Gal(β 1- |
| | 4)Glc-B- |
| NOR2 | Gal(α 1-4)GalNAc-(β 1-3)Gal(α 1-4)GalNAc(β 1- |
| | 3)Gal(α 1-4)Gal(β 1-4)Glc-B- |
| NOT int | GalNAc(β 1-3)Gal(α 1-4)GalNAc(β1-3)Gal-(α 1- |
| | 4)Gal(β 1-4)Glc-B- |
| O antigen | Fuc (α 1-2)Gal(β1-3)GlcNAc(β1-3)Gal-B-, |
| OAc-GT1b | Neu5Ac(α 2-3)DGal(β1-3)DGalNAc(β1- |
| | 4)XNeu5Ac9Ac(α 2-8)Neu5Ac(α 2-3)]DGal(β- |
| | 4)DGlc(β1-1)-B- |
| P antigen (Gb4) | Gal(α 1-4)Gal-(β 1-4)GlcNAc(β 1-3)Gal(β 1- |
| | 4)Glc-B- |
| Pk Antigen (Gb3) | Gal(α 1-4)Gal(β1-4)Glc-B- |
| Pi | Galα1-4Galβ1-4GlcNAcβ-B- |
| Schisto | GalNAcβ1,4Glcβ-B- |
| Sialyl Lewis a, | Neu5Ac(α 2-3)Gal(β1-3)[Fuc(α 1-4)]GlcNAc(β1-)B- |
| Sialyl Lewis c | Neu5Ac(α 2-3)Gal(β1-3)GlcNAc(β1-)B- |
| Sialyl Lewis x, | Neu5Ac(α 2-3)Gal(β1-4)[Fuc(α 1-3)]GlcNAc(β1- |
| | 3)Gal(β1-4)GlcNAc(β1-3)Gal(β1-)B- |
| Sialyl Lewis x, | Neu5Ac(α 2-3)Gal(β1-4)[Fuc(α 1-3)]GlcNAc(β1-)B- |
| Sialyl Lewis x-i | Neu5Ac(α 2-3)Gal(β1-4)[Fuc(α 1-3)]GlcNAc(β1- |
| | 3)Gal(β1-4)GlcNAc(β1-3)Gal(β1-)B- |
| LNT | Galβ1-3GlcNAcβ1-3Galβ1-4Glcβ-B- |
| LNnT | Galβ1-4GlcNAcβ1-3Galβ1-4Glcβ-B- |
| Sialyl-TF | Neu5Ac(α 2-6) Gal (β1-3) α GalNAc-B-, |
| sialyl-Tn | Neu5Ac(α 2-6)GalNAc-B-, |
| sLac | NeuAc-Gal0-3GicNAcB-3GalB-4Glc-B- |
| Spirometo | Galβ1,4Glcβ1,3Galβ-B- |
| Sulfatide: | Sulfate3Galβ1-B- |
| TF/Core-1 | α Gal (β1-3)aGalNAc-B-, |
| 3′-sialyl-TF | Neu5Acα2-3Galβ1-3GalNAcα-B- |
| Tn | α GalNAc-B- |
| 6-SiaβTF | Neu5Acβ2-6(Galβ1-3)GalNAcα-B- |
| 3-LacNAc-Tₙ | Galβ1-4GlcNAβ1-3GalNAcα-B- |
| 6-LacNAc-Tₙ | Galβ1-4GlcNAβ1-6GalNAcα-B- |
| 6′SLN | Neu5Acα2-6Galβ1-4GlcNAcβ-B- |
| Core 2 | GlcNAcβ1-6(Galβ1-3)GalNAcα-B- |
| Core 4 | GlcNAcβ1-3(GlcNAcβ1-6)GalNAcα-B- |
| Trifucosyl-Lewis b Antigen | Fuc(α 1-2)Gal(β1-3)[Fuc(α 1-4)]GlcNAc(b1-3)Gal(β1- |
| | 3)[Fuc(α 1-4)]GlcNAc(β1-)B- |
| Trifucosyl-Lewis y Antigen | Fuc(α 1-2)Gal(β1-4)[Fuc(α 1-3)]GlcNAc(b1-3)Gal(β1- |
| | 4)[Fuc(α 1-3)]GlcNAc(β1-)B- |
| Type 1 | GalNAc(α 1-3)[Fuc(α 1-2)]Gal(β1-3)GlcNAc[Fuc(α 1- |
| | 4)]-B-, |
| Type 1 A | GalNAc(α1-3)[Fuc(α 1-2)]Gal(β1-3)GlcNAc(β1-3)Gal- |
| | B-, |
| Type 2 A | GalNAc(α1-3)[Fuc(α 1-2)]Gal(β1-4)GlcNAc-B-, |
| Type 3 A | GalNAc(α1-3)[Fuc(α 1-2)]Gal(β1-3)GalNAc(β1- |
| | 3)Gal[Fucα 1-2)-B-, |
| Type 4 A | GalNAc(α1-3)[Fuc(α 1-2)]Gal(β1-3)GalNAc(β1-3)Gal(a |
| | 1-4)Gal(β 1-4)Glc-B-, |
| VIM-2 | Neu5Ac(α 2-3)Gal(β1-4)GlcNAc(β1-3)Gal(β1-4)[Fuc(α- |
| | 3)]GlcNAc(β1-)B- |
| Type 4 A | GalNAc(α1-3)[Fuc(α 1-2)]Gal(β1-3)GalNAc(β1-3)Gal(a |
| | 1-4)Gal(β 1-4)Glc-B-, |
| Man₃ | Manα1-3(Manα1-6)Manα-B- |
| 3′SLN | Neu5Acα2-3Galβ1-4GlcNAcβ-B- |
| 6′SL | Neu5Acα2-6Galβ1-4Glcβ-B- |

wherein B is a linker or absent;
optionally R₃ may be a polysaccharide which comprises two or more polysaccharides selected from i) to v) connected to each other.

5. The polysaccharide complex according to claim 1 or 2 wherein the heterologous lipid carrier is selected from the group
i) Monosialotetrahexosyiganglioside (GM1), wherein in formula (I),
R₁ is —(C₁₆)alkyl;
R₂ is —CH═CH(CH₂)ᵣCH₃;
R₃ is βDGal(1-3) βDGalNAc(1-4)[αNeu5Ac(2-3)] βDGal(1-4) βDGlc(1)-;
r is 12; or
ii) Monosialotetrahexosylganglioside red (GM1red), wherein in formula (I),
R₁ is —(C₁₆)alkyl;
R₂ is —CH═CHCH₂OH;
R₃ is βDGal(1-3) βDGalNAc(1-4)[αNeu5Ac(2-3)] βDGal(1-4) βDGlc(1)-;
iii) Globotriaosylceramide (Gb3), wherein in formula (I).
R₁ is —(C₁₇)alkyl
R₂ is —CH═CH(CH₂)ᵣCH₃
r is 12
R₃ is αD-Gal(1-4) βDGal(1-4) βD-Glc-
iv) a GM1-Gb3 chimera having the formula (III) 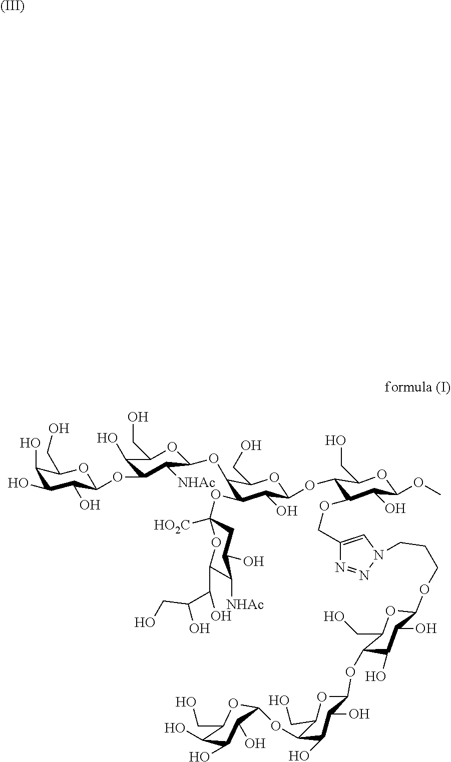
wherein in formula (I)
R₁ is —(C₁₆)alkyl or —(C₁₇)alkyl;
R₂ is —CH═CHCH₂OH or —CH═CH(CH₂)ᵣCH₃;
r is 12
R₃ is the non-lipid part of formula (III);
v) Ganglioside GD1a, wherein in formula (I),
R₁ is —(C₁₆)alkyl;
R₂ is —CH═CH(CH₂)ᵣCH₃;
r is 12;
R₃ is αNeu5Ac(2-3)βDGal(1-3)βDGalNAc(1-4)[αNeu5Ac(2-3)]βDGal(1-4)βDGlc(1)-
or
vi) a Ganglioside selected from the group consisting of asialo-GM1, asialo-GM2, GM2, GM3, GM4, GD2, GD3, GD1b, GT1b, GT1c, GT3, GQ1c, GA1, GA2, GM1b, GalCer, GalNAc-GD1a, GbOse3Cer, GbOse4Cer, GD1b-lactone, GD1c, GD1a, GGal, GlcCer, Globo, Globo-H, Gb5, monosialyl-Gb5, disialyl-Gb5, iso-Gb3, iso-Gb4, Forssman, GM1a, GM1b, GM2b, GP1c, GP1cα, GQ1b, GQ1bα, GT1a, GT1α, GT2, Internal Lewis x, Isoglobo, LacCer, Lacto, Lewis a, Lewis b, Lewis x, Lewis y, Mollu, Muco, N-Acetyl GD3, Neogala, Neolacto, N-Glycolyl GM3, 3′-SL, NOR1, NOR2, OAc-GT1b, Gb4, Gb3, Schisto, Sialyl Lewis a, Sialyl Lewis c, Sialyl Lewis x, Sialyl Lewis x-i, LNT, LNnT, Sialyl-TF, sialyl-Tn, sLac, Spirometo, Sulfatide, TF/Core-1, 3′-sialyl-TF, 3′-sialyl-TF, Tn, 6-SiaβTF, 3-LacNAc-Tₙ, 6-LacNAc-Tₙ, 6′SLN, Core 2, Core 4, Trifucosyl-Lewis b Antigen, Trifucosyl-Lewis y Antigen, Type 1, Type 1 A, Type 2 A, Type 3 A, Type 4 A, VIM-2, 3′SLN, 6′SL.

6. A method for preparing a polysaccharide complex, comprising the step of
a) combining the polysaccharide (P1) and a heterologous lipid carrier as defined in claims 1 to 5 in a solution.

7. The method of claim 6, wherein
i) the polysaccharide (P1) is autoclaved before combining with the heterologous lipid carrier preferably at a temperature of 80 to 150° C., more preferably at 95 to 140° C., most preferably at 110 to 130° C. and/or
ii) wherein the polysaccharide (P1) and the heterologous lipid carrier are combined at a temperature of at least 37° C., preferably of at least 50° C., more preferably of at least 60° C., most preferably of at least 65° C., particularly preferred of at least 70° C. for 2 to 8 h, preferably for 2.5 to 6 h, more preferably for 2.8 to 4 h or wherein the polysaccharide (P1) and the heterologous lipid carrier are combined at a temperature of at least 26 to 45° C., preferably of at least 30 to 40° C., more preferably 35 to 40° C. for at least 10 h, preferably for at least 12 h.

8. The polysaccharide complex of claims 1 to 5 or the method of claim 6 or 7, wherein
i) in the ratio between the most frequent binding type in the polysaccharide (P1) between the monosaccharides and the less frequent binding type between monosaccharides is at least 15:1, preferably at least 50:1, more preferably at least 100:1; or there is only one binding type between the monosaccharides and/or
ii) the polysaccharide (P1) comprises at least one polysaccharide selected from the group consisting of starch, amylose, amylopectin, cellulose, glycogen, chitosan, methylmannose; preferably the polysaccharide (P1) comprises amylose and branched amylopectin molecules, more preferably the polysaccharide (P1) comprises amylose and branched amylopectin molecules in molar ratios of 15%-25% amylose and 85%-75% branched amylopectin molecules or starch and/or
iii) at least 80% of the heterologous lipid carrier associated with the polysaccharide (P1) remains associated after a treatment with 0.3% bile salts, optionally in combination with pancreatine juice in PBS or DPBS for at least 1 hour at least 37° C.

9. A polysaccharide complex preparable by the method of claim 6 or 7.

10. A composition comprising
i) one or more of the polysaccharide complex(es) of claims 1 to 5 and 8; preferably said composition comprises a mixture of same or different polysaccharide complex(es) of claims 1 to 5 and 8 and/or
ii) one or more of the polysaccharide complex(es) of claims 1 to 5 and 8 and at least one pharmaceutically acceptable carrier.

11. The polysaccharide complex of claims 1 to 5, 8 and 9 or the composition of claim 10
A) for use in medicine or
B) for use in the treatment of a subject comprising
i) binding and/or reducing toxicity of and/or neutralizing a toxin;
ii) binding and/or reducing the pathogenicity and/or neutralizing a pathogenic microorganism;
iii) binding a receptor of a toxin;
iv) binding a receptor of a pathogenic microorganism;
v) eliciting or modulating an immune response;
vi) preventing disease;
vii) monitoring the development of a disease and/or assessing the efficacy of a therapy of a disease;
viii) delivering a pharmaceutically active compound, preferably said delivering is to a mucosal tissue of a subject; or
C) in an in vivo or in vitro method of diagnosis.

12. The composition of claim 10 or 11, wherein said composition is suitable for oral, enteral, dermal, topical, urogenital, inhalational administration.

13. Use of the composition of claim 10 or 11 as nutraceutical.

14. A vaccine or adjuvant comprising the polysaccharide complex or composition according to any one of preceding claims.

15. The vaccine or adjuvant comprising the polysaccharide complex or composition of claim 14, wherein said vaccine or adjuvant is suitable for oral or enteral administration.
